(19) United States
(12) Patent Application Publication
(10) Pub. No.: US 2011/0082312 A1
(43) Pub. Date: Apr. 7, 2011

(54) PRODUCTION OF COMPOUNDS COMPRISING CF30 GROUPS

(75) Inventors: Nikolai (Mykola) Ignatyev, Duisburg (DE); Wolfgang Hierse, Gross-Zimmern (DE); Martin Seidel, Darmstadt (DE); Andreas Bathe, Darmstadt (DE); Jens Schroeter, Gross-Umstadt (DE); Karsten Koppe, Marl-Polsum (DE); Thorsten Meier, Duesseldorf (DE); Peter Barthen, Rheinberg (DE); Walter Frank, Wuppertal (DE)

(73) Assignee: MERCK PATENT GESELLSCHAFT MIT DESCHRANKTER HAFTUNG, Darmstadt (DE)

(21) Appl. No.: 12/992,959

(22) PCT Filed: May 4, 2009
(86) PCT No.: PCT/EP2009/003177

§ 371 Date: Nov. 16, 2010

(30) Foreign Application Priority Data
May 19, 2008 (DE) 10 2008 024 221.7

(51) Int. Cl. C07C 69/025; C07C 41/01; C07C 43/15; C07C 67/31; C07C 43/12; C07C 51/09; C07C 41/26; C07C 45/64; C07C 49/303; C07C 43/14

(52) U.S. Cl. 560/184; 568/685; 568/684; 568/681; 568/674; 568/676; 562/586; 568/323; 568/336

(57) ABSTRACT

The present invention relates to a process for the preparation of compounds containing CF₃O groups using compounds containing at least one group Y, in which Y=—Hal, —OSO₂(CF₂)_{z}F, —OSO₂C_{z}H_{2z+1} (z=1-10), —OSO₂F, —OSO₂Cl, —OC(O)CF₃— or —OSO₂Ar, to a process for the preparation of compounds containing CF₃O groups using KOCF₃ and/or RbOCF₃, and to novel compounds containing CF₃O groups, and to the use thereof.

The present invention relates to a process for the preparation of compounds containing CF₃O groups using compounds containing at least one group Y, in which Y=—Hal, —OSO₂(CF₂)_{z}F, —OSO₂C_{z}H_{2z+1} (z=1-10), —OSO₂F, —OSO₂Cl, —OC(O)CF₃— or —OSO₂Ar, to a process for the preparation of compounds containing CF₃O groups using KOCF₃ and/or RbOCF₃, and to novel compounds containing CF₃O groups, and to the use thereof.

In organic chemistry, the CF₃O group has been known for a long time. The first compounds containing CF₃O groups were discovered more than 50 years ago. Thus, for example, phenyl trifluoromethyl ether was synthesised by L. M. Yagupolskii in 1955 by fluorination of the corresponding phenyl trichloromethyl ether using SbF₃ in the presence of SbCl₅. The standard method for the introduction of CF₃O groups into aromatic rings was developed in 1964 by W. A. Sheppard. This method is based on the fluorination of arylfluoroformates, which are formed by reaction of phenols with difluorophosgene, by means of sulfur tetrafluoride (W. A. Sheppard, J. Org. Chem., Vol. 29, 1964, No. 1, pp. 1-11).

The most important method in industry was developed by A. Feiring and gives the corresponding aryl trifluoromethyl ethers in good yields by reaction of phenols with CCl₄ in HF at 100-150° C. in an autoclave (A. Feiring, J. Org. Chem., Vol. 44, 1979, No. 16, pp. 2907-2910).

However, this reaction is not suitable for the preparation of alkyl trifluoromethyl ethers. Another method, which is based on the fluorination of dithiocarbonates (xanthate esters) by means of HF/pyridine complex in the presence of 1,3-dibromo-5,5-dimethylhydantoin (DBH) (J.-C. Blazejewski et al. J. Org. Chem., 66 (2001), pp. 1061-1063), has been developed for the preparation of these compounds.

The disadvantages of this method are the use of the hazardous HF/pyridine complex, the preparation of xanthate esters using toxic and flammable CS₂, and the large amounts of waste.

Another method for the introduction of the CF₃O group into organic molecules is based on the use of CF₃O⁻ salts (R. Minkwitz et al. Z. Naturforsch., 51b (1996), pp. 147-148; A. A. Kolomeitsev et al. Tetrahedron Letters, 49 (2008), pp. 449-454).

The synthesis of compounds containing CF₃O groups which contain readily derivatisable groups, such as halogens or double bonds, is not described by this method.

Only the preparation of allyl-OCF₃ in 29% yield by an autoclave reaction at 50° C. in diglyme from allyl bromide, COF₂ and KF is known (JP 03264545 A).

WO 2006/072401 describes compounds which carry at least one terminal trifluoromethoxy group and contain a polar end group, are surface-active and are excellently suitable as surfactants.

The object of the present invention is therefore to provide an alternative process for the preparation of compounds containing CF₃O groups which is simple and economical to carry out. Furthermore, the object of the present invention is to provide novel compounds containing CF₃O groups.

This object is achieved by a process for the preparation of compounds containing at least one CF₃O group, comprising the reaction of CF₃O⁻ salts with the compounds of the formula (I) according to the invention according to Claim 1, and by the compounds of the formula (II) according to Claim 9.

The present invention relates firstly to a process for the preparation of compounds containing at least one CF₃O group, comprising at least the reaction of CF₃O⁻ salts with compounds of the formula (I)

XₘCHₙ(LₒY)ₚ (I)

where:

X=—Cl, —Br, —I, —OR, —SR, —C(O)R, —C(O)OR, —H, —CN, —CR¹═CR²₂, —C≡CR² or —(CR³R⁴)_{q}X,

Y=—Hal, —OSO₂(CF₂)_{z}F, —OSO₂C_{z}H_{2z+1}, —OSO₂F, —OSO₂Cl, —OC(O)CF₃ or —OSO₂Ar,

L=independently of one another a single bond or a linear or branched (CR³R⁴)_{q}-alkyl, optionally containing at least one aromatic ring, cycloalkyl, heterocyclic ring, O atom, S atom, double bond, triple bond and/or group X in the chain and/or in the side chain,

Ar=substituted or unsubstituted aryl, preferably aryl which is substituted by —CH₃, —NO₂, or —Br,

m=1-2

n=0-2

o=0 or 1

p=1-3

q=1-20, preferably 1-12

m+n+p=4

z=1-10, preferably 1-4

R=aryl or cycloalkyl or alkylaryl (for example benzyl), optionally substituted by at least one fluorine and/or chlorine and/or bromine and/or iodine atom and/or other functional group (such as, for example, NO₂, NH₂, CN, C(O)R, C(O)OR, C(O)NR₂), linear or branched H(CR³R⁴)ᵣ-alkyl (r=1 to 20), optionally containing at least one aromatic ring, heterocyclic ring, O atom, S atom, double bond and/or triple bond and optionally substituted by at least one fluorine and/or chlorine atom,

R¹, R², R³ and R⁴=independently of one another H, aryl, cycloalkyl, optionally substituted by at least one fluorine and/or chlorine atom, linear or branched alkyl, optionally containing at least one aromatic ring, heterocyclic ring, O atom, S atom, double bond and/or triple bond and optionally substituted by at least one fluorine and/or chlorine atom, and CHₙ and Lₒ may together form a cycloalkyl or aromatic ring or hetero-cyclic ring.

In a variant of the invention, compounds of the formula (I) in which n=2, o=0 or 1, in particular 1, and p=1 are preferably used.

It is likewise preferred to use compounds of the formula (I) in which Y=—Hal, —OSO₂CF₃, —OSO₂CH₃, —OSO₂F or —OSO₂Ar, in particular Y=—Hal, —OSO₂Ar, or —OSO₂CH₃. Compounds where Y=−I, —OSO₂Ar or —OSO₂CH₃, in particular Y=—OSO₂CH₃, are particularly advantageous.

Another preferred embodiment of the present invention is the use of compounds of the formula (I) in which X=—Cl, —Br, —I, —OR, —C(O)OR or —CR¹═CR²₂. Particularly preferably, X=—Br, —OR or —CR¹═CR²₂.

It is additionally preferred to use compounds of the formula (I) in which L=linear or branched (CR³R⁴)_{q}-alkyl, which optionally contains at least one O atom, in particular compounds where R³ and R⁴=independently of one another H and/or linear or branched C1-C6 alkyl.

R¹ and R² are preferably, independently of one another, H or methyl.

R³ and R⁴ are preferably, independently of one another, H or linear or branched C1-C6 alkyl or cycloalkyl.

A preferred embodiment of the process according to the invention consists in the use of compounds which contain combinations of the above-mentioned preferred variables, in particular those in which all preferred forms of the variables occur.

Particular preference is given to the use of compounds of the formula (I) in which

n=2, o=0 or 1, in particular 1, p=1,

Y=−Hal, —OSO₂CF₃, or —OSO₂CH₃, in particular —OSO₂CH₃

X=—Cl, —Br, —C(O)OR, or —CR¹═CR²₂,

L=linear or branched (CR³R⁴)_{q}-alkyl, optionally containing at least one O atom,

R¹ and R²=independently of one another H or methyl, and

R³ and R⁴=independently of one another H and/or linear or branched C1-C6 alkyl or cycloalkyl.

The compounds of the formula (I) to be used in accordance with the invention may contain the groups Y once, twice or three times, preferably once. However, the groups Y may also be present multiple times.

For the reaction of the compounds of the formula (I) according to the invention, all known CF₃O⁻ salts can be used. Suitable salts are, for example, those with the cations K⁺, Rb⁺, Cs⁺, (R⁵)₄N⁺, in which R⁵ can be, independently of one another, C₁-C₄ alkyl, the cation of DFI (=DFI without F; DFI=2,2-difluoro-1,3-dimethylimidazolidine (CAS 220405-40-3)) or tris(dimethylamino)sulfonium cation (as guanidinium salt). In a variant of the invention, (R⁵)₄N⁺CF₃O⁻ salts in which R⁵ can be, independently of one another, C1-C4-alkyl, in particular C1-C2-alkyl, are used.

The present invention furthermore relates to a process for the preparation of compounds containing at least one CF₃O group, comprising the use of KOCF₃ and/or RbOCF₃. KOCF₃ and RbOCF₃ can be formed in situ. RbOCF₃ can also be added separately. Particular preference is given to the use of RbOCF₃. Surprisingly, it has been found that RbOCF₃ (formed in situ or as substance) gives better results in the reaction of allyl bromide or iodide, in particular of allyl iodide, compared with Cs⁺CF₃O⁻ salts (the proportion of the by-product allyl fluoride is low) and does not require a complex autoclave technique.

KOCF₃ and/or RbOCF₃ is/are reacted, in particular, with compounds containing at least one group Y in which Y=—Hal, —OSO₂CF₃, —OSO₂CH₃, —OSO₂F, —OSO₂Cl, —OC(O)CF₃, or —OSO₂Ar.

Preference is given to the reaction with compounds containing at least one group Y, in which Y=—Hal, —OSO₂CF₃, —OSO₂CH₃ or —OSO₂Ar. Particular preference is given to compounds where Y=—I, —OSO₂CH₃ or —OSO₂Ar. Y=—OSO₂CH₃ is especially suitable for the purposes of the invention. In combination with potassium iodide, the use of Y=Br or Cl is also possible.

KOCF₃ and/or RbOCF₃ are preferably used in combination with compounds of the formula (I), in particular in combination with compounds of the formula (I) where

n=2, o=0 or 1, in particular 1, and p=1, q=1

Y=Hal, —OSO₂CF₃, or —OSO₂CH₃,

X=Cl, Br, I, or —CR¹═CR²₂,

L=linear or branched (CR³R⁴)_{q}-alkyl, optionally containing at least one O atom,

R¹ and R²=independently of one another H or methyl, and

R³ and R⁴=independently of one another linear or branched C1-C6 alkyl. Particular preference is given to the reaction of RbOCF₃ with compounds of the formula (I) in which X=—CR¹═CR²₂, —Hal or —OR, in particular —CR¹═CR²₂, and Y=—OSO₂CH₃.

In the processes according to the invention, the reactions of the compounds of the formula (I) with the CF₃O⁻ salts are preferably carried out at temperatures of −30 to 120° C., particularly preferably 0° C. to 100° C., very particularly preferably room temperature to 80° C. Depending on the reactivity of the X and Y groups and of the solvent, temperatures between 0° C. and 80° C. are necessary. The preferred temperature in the case of particularly reactive X groups (for example —OSO₂CF₃) is between 0° C. and 30° C. In the case of less reactive X groups, the temperature is between 30 and 80° C.

The reaction times are dependent on the reactivity of the reactants employed. They are between 1 hour and up to 36 hours, depending on the case.

The processes according to the invention can be carried out at atmospheric pressure. It is not necessary to carry out the reaction under increased pressure, for example in an autoclave.

Preferred solvents for the reactions with CF₃O⁻ salts, in particular both with R⁵₄N⁺CF₃O⁻ salts and also with KOCF₃ or RbOCF₃, are organic solvents, particularly preferably polar aprotic solvents. The reaction can be carried out in acetonitrile, N,N-dimethylformamide, N,N-dimethylacetamide, N-methylpyrrolidone and/or other amides of secondary amines. Particular preference is given to N,N-dimethylformamide, N,N-dimethylacetamide and/or N-methylpyrrolidone.

The purification of the compounds of the formula (II) is possible by methods familiar to the person skilled in the art, such as filtration, extraction with solvents and/or (fractional) distillation, optionally under reduced pressure.

The present invention furthermore also relates to a process for the preparation of KOCF₃ and RbOCF₃ in which KF or RbF is reacted with trifluoromethyl triflate or difluorophosgene, as illustrated by way of example for RbOCF₃ in the following scheme. The preparation by means of trifluoromethyl triflate is particularly preferred.

CF₃SO₂OCF₃+RbF→RbOCF₃+CF₃SO₂F↑COF₂+RbF→RbOCF₃

The reaction is preferably carried out at temperatures of −60° C. to 30° C., particularly preferably −30° C. to room temperature, very particularly preferably at −20° C. to 0° C.

Preferred solvents for the preparation of KOCF₃ and RbOCF₃ are organic solvents, particularly preferably polar aprotic solvents. The reaction can be carried out in acetonitrile, N,N-dimethylformamide, N,N-dimethylacetamide, N-methylpyrrolidone and/or other amides of secondary amines. N,N-dimethylformamide, N,N-dimethylacetamide and/or N-methylpyrrolidone are particularly preferred.

The present invention furthermore relates to compounds of the formula (II)

XₘCHₙ(LₒOCF₃)ₚ (II)

where

X=—Cl, —Br, —I, —OR, —SR, —C(O)R, —C(O)OR, —H, —CN, —CR¹═CR²₂, —C≡CR² or —(CR³R⁴)_{q}X,

L=independently of one another a single bond or linear or branched (CR³R⁴)_{q}-alkyl, optionally containing at least one aromatic ring, cycloalkyl, heterocyclic ring, O atom, S atom, double bond, triple bond, and/or group X in the chain and/or in the side chain,

m=1-2

n=0-2

o=1

p=1-3

q=2-20

m+n+p=4

R=aryl or cycloalkyl or alkylaryl (for example benzyl), optionally substituted by at least one fluorine and/or chlorine and/or bromine and/or iodine atom and/or other functional group (such as, for example, NO₂, NH₂, CN, C(O)R, C(O)OR, C(O)NR₂), linear or branched H(CR³R⁴)ᵣ-alkyl (r=1 to 20), optionally containing at least one aromatic ring, heterocyclic ring, O atom, S atom, double bond and/or triple bond and optionally substituted by at least one fluorine and/or chlorine atom,

R¹, R², R³ and R⁴=independently of one another H, aryl, cycloalkyl, optionally substituted by at least one fluorine and/or chlorine atom, linear or branched alkyl, optionally containing at least one aromatic ring, heterocyclic ring, O atom, S atom, double bond and/or triple bond and optionally substituted by at least one fluorine and/or chlorine atom,

and CHₙ and Lₒ may together form a cycloalkyl or aromatic ring or hetero-cyclic ring,

where the compounds CF₃O—(CH₂—CH₂—O)₂—OCF₃ and C₂H₅O—(CH₂—CH₂—O)₂—C₂H₅—OCF₃ are excluded.

In a preferred variant of the present invention, X=—Cl, —Br, —I, —C(O)OR or —CR¹═CR²₂, o=1 and L=branched (CR³R⁴)_{q}-alkyl, optionally containing at least one O atom, R¹ and R²=independently of one another H or methyl, and R³ and R⁴=independently of one another H and/or linear or branched C1-C6 alkyl or cycloalkyl.

Preferred compounds of the formula (II) are also those in which X=H.

Other preferred compounds of the formula (II) are those which contain no further fluorinated groups apart from CF₃O groups.

In another variant, X=—Cl, —Br, —I, —OR, —CR¹═CR²₂ or —(CR³R⁴)_{q}X, L=independently of one another linear or branched (CR³R⁴)_{q}-alkyl, containing at least one aromatic ring, cycloalkyl, heterocyclic ring, S atom, triple bond, and/or group X, apart from X=halogen, in the chain and/or in the side chain.

The compounds of the formula (II) according to the invention contain the OCF₃ group once, twice or three times, preferably once. However, the OCF₃ group may also be present multiple times. Examples of such structures are the following compounds 3-trifluoromethoxy-2-trifluoromethoxymethylpropene (A), 1,3-bistrifluoromethoxypropan-2-one (B), 6-trifluoromethoxy-5-trifluoromethoxymethylhex-1-ene (C) and (2-benzyloxymethyl-6-trifluoromethoxyhexyloxymethyl)benzene (D).

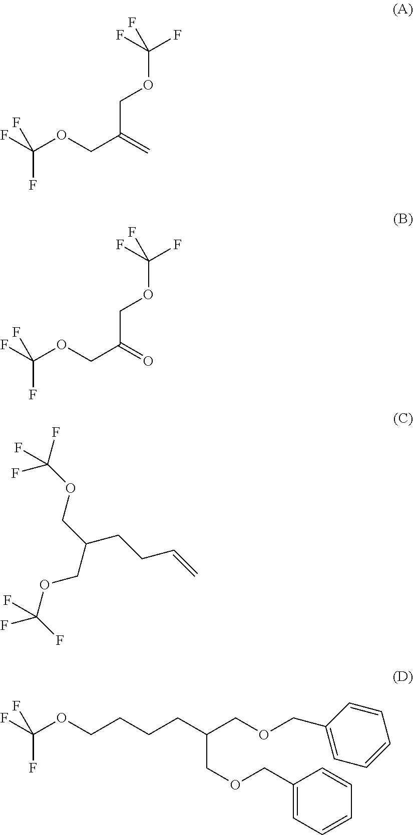

The compounds of the formula (I) according to the invention can be prepared by processes known to the person skilled in the art. In particular, the reaction of alcohols of the formula (III)

XₘCHₙ(LₒOH)ₚ (III)

in which the variables have the meanings given for the formula (I), in particular also the preferred meanings and the combinations of the preferred embodiments,

with (CF₃SO₂)₂O, C_{z}H_{2z+1}SO₂Cl, ClSO₂Cl, ClSO₂F, FSO₂F, (CF₃CO)₂O or ArSO₂Cl is suitable for the preparation of the compounds of the formula (I).

Compounds of the formula (III) where X=—Cl, —Br, —C(O)OR or —CR¹═CR²₂ are preferably used.

In addition, compounds of the formula (III) are preferably reacted with (CF₃SO₂)₂O, CH₃SO₂Cl, ClSO₂F or ArSO₂Cl (for example 4-Me—C₆H₄—SO₂Cl).

For the synthesis of the compounds of the formula (I), the corresponding alcohols of the formula (III) are reacted with the respective reagents. This is described below by way of example for compounds where X=—CH═CH₂ and L=—(CH₂)_{q}-, where q is preferably=1-20. The following reaction schemes also reproduce the further reaction to give the compounds containing CF₃O groups for the example of RbOCF₃.

Preparation of the Triflate Compounds and Reaction Thereof with RbOCF₃:

CH₂═CH(CH₂)ₙOH+(CF₃SO₂)₂O+C₅H₅N→CH₂═CH(CH₂)ₙOSO₂CF₃+C₅H₅NH⁺⁻OSO₂CF₃

CH₂═CH(CH₂)ₙOSO₂CF₃+RbOCF₃→CH₂═CH(CH₂)ₙOCF₃+RbOSO₂CF₃

The by-product pyridinium triflate is preferably removed after the first reaction step.

Preparation of the Trifluoroacetate Compounds and Reaction Thereof with RbOCF₃:

CH₂═CH(CH₂)ₙOH+(CF₃CO)₂O+C₅H₅N→CH₂═CH(CH₂)ₙOC(O)CF₃+C₅H₅NH⁺⁻O(O)CF₃

CH₂═CH(CH₂)ₙOC(O)CF₃+RbOCF₃→CH₂═CH(CH₂)ₙOCF₃+RbOC(O)CF₃

### Preparation of the Mesylate Compounds:

CH₂═CH(CH₂)ₙOH+CH₃SO₂Cl+(C₂H₅)₃N→CH₂═CH(CH₂)ₙOSO₂CH₃+(C₂H₅)₃N.HCl

The HCl formed is preferably removed, for example by addition of a base, such as pyridine or triethylamine, or by complexing with dioxane and distillative removal of the complex formed.

This reaction can also be carried out in the presence of KF, which not only replaces Cl with F, but also as a base scavenges HF.

CH₂═CH(CH₂)ₙOH+CH₃SO₂Cl+2 KF→CH₂═CH(CH₂)ₙOSO₂CH₃+KCl+KHF₂

### Preparation of the Chloro- or Fluorosulfonyl Compounds:

CH₂═CH(CH₂)ₙOH+ClSO₂Cl+3 KF→CH₂═CH(CH₂)ₙOSO₂F+2 KCl+KHF₂

CH₂═CH(CH₂)ₙOH+ClSO₂Cl+C₄H₈O₂→CH₂═CH(CH₂)ₙOSO₂Cl+C₄H₈O₂.HCl

The synthesis of the compounds of the formula (I) containing other groups X, in particular X=Cl, Br or I, can be carried out analogously to the above reactions.

The compounds of the formula (II) according to the invention can be employed in various synthetic processes for the preparation of organic compounds containing CF₃O groups. They are suitable, for example, for hydrolyses, nucleophilic substitutions, oxidations, epoxidations, hydrogenations, hydroborations with subsequent oxidation, metathesis of olefins, and other reactions known to the person skilled in the art. The compounds according to the invention can be used, in particular, for the preparation of compounds of the general formulae (IV), (V) and (VI), where L and X have the meanings described above and X may, in particular, also be an OH group.

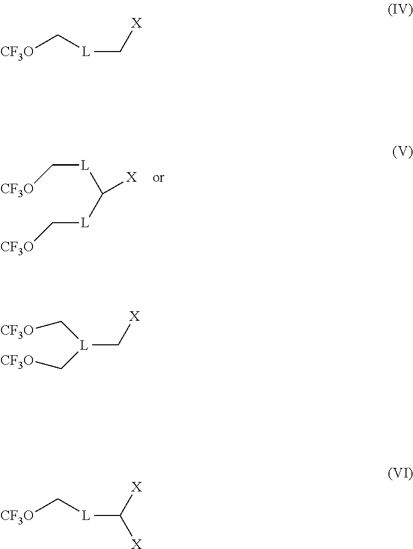

The compounds of the formula (II) can preferably be used for the preparation of surface-active compounds containing CF₃O groups. Such compounds are used as interface promoter or emulsifier, in particular in the preparation and use of fluoropolymers. Further applications of the compounds according to the invention are described in WO 2006/072401.

It may be particularly advantageous in the present invention, in particular, that an autoclave technique is not necessary, that the yields can be improved and that favourable and commercially available starting materials can be used. A further advantage is the use of numerous starting materials, in particular those of the formula (I), since the present process is not restricted only to allyl compounds. It is particularly advantageous that the rubidium salt can be regenerated. These advantages were unexpected and unpredictable for the person skilled in the art. The present invention is also particularly suitable for large-scale industrial productions, since it is an efficient and economical process.

Apart from the preferred compounds, the use thereof, agents and processes mentioned in the description, further preferred combinations of the subject-matters according to the invention are disclosed in the Claims.

The disclosures in the literature references cited hereby expressly also belong to the disclosure content of the present application.

The following examples explain the present invention in greater detail without restricting the scope of protection. In particular, the reaction conditions, features, properties and advantages, described in the examples, of the compounds on which the relevant examples are based can also be applied to other substances and compounds which are not mentioned in detail, but fall within the scope of protection, unless stated otherwise elsewhere. In addition, the invention can be carried out throughout the range claimed and is not restricted to the examples given here.

### EXAMPLES

### Example 1

### Allyl trifluoromethyl ether

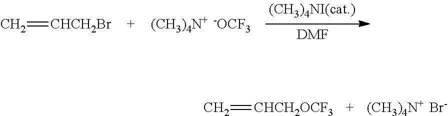

31.4 g (197 mmol) of tetramethylammonium trifluoromethoxylate and 3.6 g (18 mmol) of tetramethylammonium iodide are suspended in 100 ml of dry dimethylformamide (DMF) in a 250 ml round-bottomed flask. 21.7 g (179 mmol) of allyl bromide are added to this suspension with stirring, and the reaction mixture is stirred at 60° C. for three days. All volatile products are then distilled off in vacuo at 10⁻² mbar and room temperature into a cooled distillation trap (−196° C.). In total, 21.5 g of a liquid material which comprises allyl-OCF₃, allyl-F, allyl-Br and allyl-I are obtained. This mixture is subjected to fractional distillation, and 16.2 g of allyl-OCF₃ are obtained. The yield of allyl trifluoromethyl ether is 72%. The product is characterised by means of ¹H- and ¹⁹F-NMR spectra.

¹H-NMR (solvent: CD₃CN; reference substance: TMS), δ, ppm: 4.56 d,m (CH₂), 5.35 d,m (CH), 5.45 d,m (CH), 5.99 m (CH); ³J_{H,H}=5.8 Hz, ³J_{H,H}=10.4 Hz, ³J_{H,H}=17.2 Hz. ¹⁹F-NMR (solvent: CD₃CN; reference substance: CCl₃F), δ, ppm: −59.7 s (OCF₃).

### Example 2a

### 1-Methylsulfonylprop-2-ene

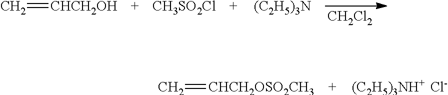

32.18 g (318.1 mmol) of triethylamine and 12.16 g (209.3 mmol) of prop-3-en-1-ol are dissolved in 250 ml of dry dichloromethane. 29.27 g (255.5 mmol) of methanesulfonyl chloride are added dropwise to the cold solution (bath temperature: 0° C.) with stirring. The reaction mixture is stirred at 0° C. for 5.5 hours and transferred into a separating funnel. It is washed once with 90 ml of cold (0° C.) water, 70 ml of cold (0° C.) 5% hydrochloric acid, 80 ml of cold (0° C.) saturated sodium hydrogencarbonate solution and 80 ml of cold (0° C.) saturated sodium chloride solution. The solution is dried using MgSO₄, filtered, and dichloromethane is evaporated. The crude product obtained is subjected to fractional distillation in vacuo at 3·10⁻³ mbar (boiling point: 43-45° C./3·10⁻³ mbar). 21.4 g (156.9 mmol) of allyl mesylate are obtained. The yield of allyl mesylate is 75%. The product, allyl mesylate, is characterised by means of the ¹H-NMR spectrum.

¹H-NMR (solvent: CDCl₃; reference substance: TMS), δ, ppm: 3.00 s (CH₃), 4.70 d (CH₂); ³J_{H,H}=6.0 Hz, 5.36 d (cis-H, CH₂); ³J_{H,H}=5.43 Hz d (trans-H, CH₂), 5.94 d,d,t (CH).

### Example 2b

### Allyl trifluoromethyl ether

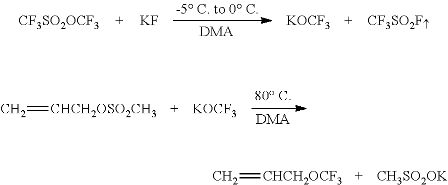

8.1 g (140 mmol) of dry potassium fluoride are suspended in 130 ml of dry N,N-dimethylacetamide (DMA) in a 250 ml round-bottomed flask with reflux condenser (cooled to −78° C.), and 29.1 g (133 mmol) of trifluoromethyl triflate, CF₃SO₂OCF₃, are slowly added with stirring and cooling of the reaction mixture by means of a bath cooled to −5° C. The reaction mixture is kept at 0° C. for 1 hour. The temperature of the reflux condenser is then increased to −20° C. in order to remove the trifluoromethylsulfonyl fluoride, CF₃SO₂F, which is formed during the reaction. 14.3 g (105 mmol) of allyl mesylate from Example 2a are added to the suspension of KOCF₃ which remains, and the reaction mixture is stirred at 80° C. for 2 hours. All volatile products are then distilled off in vacuo at 20 mbar and 80° C. into a cooled distillation trap (−196° C.). In total, 9.5 g of a liquid material which comprises 96% of allyl-OCF₃ and 4% of N,N-dimethylacetamide are obtained. The yield of allyl trifluoromethyl ether is 69%. The product is characterised by means of ¹H- and ¹⁹F-NMR spectra. The NMR data for the product, allyl trifluoromethyl ether, are identical to the data described in Example 1.

### Example 3

### Allyl trifluoromethyl ether

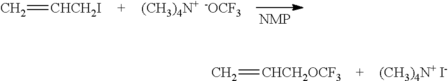

35.7 g (224 mmol) of tetramethylammonium trifluoromethoxylate are suspended in 100 ml of dry N-methylpyrrolidone (NMP) in a 250 ml round-bottomed flask. 33.9 g (202 mmol) of allyl iodide are added to this suspension with stirring, and the reaction mixture is stirred at 60° C. for 40 hours. All volatile products are then distilled off in vacuo at 10⁻¹ mbar and room temperature into a cooled distillation trap (−196° C.). In total, 21.2 g of a liquid material which comprises allyl-OCF₃, allyl-F and allyl-I are obtained. This mixture is subjected to fractional distillation, and 17.3 g of allyl-OCF₃ are obtained. The yield of allyl trifluoromethyl ether is 68%. The product is characterised by means of ¹H- and ¹⁹F-NMR spectra. The NMR data are identical to the data in Example 1.

### Example 4

### Rubidium trifluoromethoxvlate, RbOCF₃

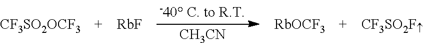

27.1 g (259 mmol) of dry rubidium fluoride are suspended in 200 ml of dry acetonitrile in a 500 ml round-bottomed flask with reflux condenser (cooled to −40° C.), and 61.7 g (283 mmol) of trifluoromethyl triflate, CF₃SO₂OCF₃, are slowly added with stirring and cooling of the reaction mixture by means of a bath cooled to −40° C. The reaction mixture is kept at −20° C. for 30 min, at 0° C. for 1 hour and at 20° C. for 30 min. The temperature of the reflux condenser is then increased to −20° C. in order to remove the trifluoromethylsulfonyl fluoride, CF₃SO₂F, which is formed during the reaction. Acetonitrile is decanted off, the residue is washed with dry acetonitrile, and the white product, RbOCF₃, is dried in vacuo at 10⁻³ mbar and at room temperature. 33.8 g of RbOCF₃ are obtained. The yield of rubidium trifluoromethoxylate is 77%, based on RbF. The product, RbOCF₃, is characterised by means of Raman spectroscopy (melting-point tube; 1274 mW).

| | | | |
|---|---|---|---|
| 1555 cm⁻¹ | ν₁ | A₁ | C—O stretching vibration |
| 809 cm⁻¹ | ν₂ | ν_{sym} | symmetrical CF₃ stretching vibration |
| 599 cm⁻¹ | ν₃ | δ_{sym} | symmetrical CF₃ deformation vibration |
| 960 cm⁻¹ | ν₄ | E | asymmetrical CF₃ stretching vibration |
| 575 cm⁻¹ | ν₅ | δ_{asym} | OCF deformation vibration |
| 422 cm⁻¹ | ν₆ | δ_{asym} | asymmetrical CF₃ deformation vibration |

This spectrum is identical to the Raman spectrum of RbOCF₃ described in the literature (K. O. Christe et al., Spectrochimica Acta, Vol. 31A, 1975, pp. 1035-1038).

### Example 5

### Potassium trifluoromethoxylate, KOCF₃

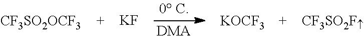

13.8 g (55 mmol) of trifluoromethyl triflate, CF₃SO₂OCF₃, are added at 0° C. to 3.1 g (53 mmol) of dry potassium fluoride in a round-bottomed flask with reflux condenser (cooled to −78° C.). 0.25 g (2.9 mmol) of N,N-dimethylacetamide (DMA) is slowly added thereto with stirring and cooling of the reaction mixture by means of a bath cooled to 0° C. The reaction mixture is kept at 0° C. for three hours. The temperature of the reflux condenser is then increased to room temperature in order to remove the trifluoromethylsulfonyl fluoride, CF₃SO₂F, which is formed during the reaction. The DMA is removed at 0° C. in vacuo, and the white product is dried in vacuo (10⁻³ mbar) at 0° C. 5.0 g of a white solid are obtained. The yield of potassium trifluoromethoxylate is 55%, based on KF. The product, KOCF₃, is characterised by means of Raman spectroscopy (melting-point tube; 892 mW).

| | | | |
|---|---|---|---|
| 1544 cm⁻¹ | ν₁ | A₁ | C—O stretching vibration |
| 812 cm⁻¹ | ν₂ | ν_{sym} | symmetrical CF₃ stretching vibration |
| 599 cm⁻¹ | ν₃ | δ_{sym} | symmetrical CF₃ deformation vibration |
| 576 cm⁻¹ | ν₅ | δ_{asym} | OCF deformation vibration |
| 421 cm⁻¹ | ν₆ | δ_{asym} | asymmetrical CF₃ deformation vibration |

### Example 6

### Allyl trifluoromethyl ether, CH₂═CHCH₂OCF₃

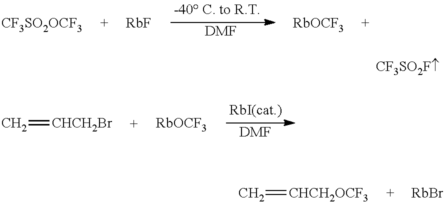

21.1 g (202 mmol) of dry rubidium fluoride are suspended in 100 ml of dry dimethylformamide (DMF) in a 250 ml round-bottomed flask with reflux condenser (cooled to −78° C.), and 47.9 g (220 mmol) of trifluoromethyl triflate, CF₃SO₂OCF₃, are slowly added with stirring and cooling of the reaction mixture by means of a bath cooled to −45° C. The reaction mixture is kept at −25° C. for 2.5 hours, at 0° C. for 1 hour and at 20° C. for 1 hour. The temperature of the reflux condenser is then increased to −20° C. in order to remove the trifluoromethylsulfonyl fluoride, CF₃SO₂F, which is formed during the reaction. 4.2 g (20 mmol) of rubidium iodide and 24.2 g (200 mmol) of allyl bromide are added to the suspension of RbOCF₃ which remains, and the reaction mixture is stirred at 60° C. for 61 hours. All volatile products are then distilled off in vacuo at 10⁻² mbar and room temperature into a cooled distillation trap (−196° C.). In total, 29.7 g of a liquid material which comprises allyl-OCF₃, allyl-F, allyl-Br and allyl-I are obtained. This mixture is subjected to fractional distillation, and 15.4 g of allyl-OCF₃ are obtained. The yield of allyl trifluoromethyl ether is 61%. The product is characterised by means of ¹H- and ¹⁹F-NMR spectra. The NMR data for the product, allyl trifluoromethyl ether, are identical to the data described in Example 1.

### Example 7

### 1-Bromo-3-(trifluoromethoxy)propane, BrCH₂CH₂CH₂OCF₃

### 7a) 1-Bromo-3-trifluoromethylsulfonylpropane (3-bromopropyl triflate)

BrCH₂CH₂CH₂OH+(CF₃SO₂)₂O+C₅H₅N→BrCH₂CH₂CH₂OSO₂CF₃+C₅H₅NH⁺⁻OSO₂CF₃

48.4 g (171.6 mmol) of trifluoromethanesulfonic anhydride are slowly added with stirring at room temperature to a solution of 13.7 g (173 mmol) of dry pyridine in 400 ml of dry dichloromethane in a 1 litre flask with reflux condenser. The suspension is stirred for a further 30 min, and 23.5 g (169 mmol) of 3-bromopropanol are then added. After the reaction mixture has been stirred at room temperature for 1 hour, it is cooled to 0° C. The salt, pyridinium triflate, is filtered off and washed with 100 ml of cold (0° C.) and dry dichloromethane. The dichloromethane solutions are combined, and CH₂Cl₂ is distilled off in vacuo. The residue is subjected to fractional distillation after further filtration. 30.5 g of liquid 3-bromopropyl triflate are obtained. The boiling point is 31-32° C. at 0.7 mbar. The yield of 3-bromopropyl triflate is 67%, based on 3-bromopropanol. The product, 3-bromopropyl triflate, is characterised by means of ¹H- and ¹⁹F-NMR spectra.

¹H-NMR (solvent: CDCl₃; reference substance: TMS), δ, ppm: 2.33 quin (CH₂), 3.48 t (CH₂), 4.68 t (CH₂); ³J_{H,H}=5.8 Hz, ³J_{H,H}=6.2 Hz. ¹⁹F-NMR (solvent: CDCl₃; reference substance: CCl₃F), δ, ppm: −74.4 s (CF₃).

### 7b) 1-Bromo-3-trifluoromethylsulfonylpropane (3-bromopropyl triflate)

BrCH₂CH₂CH₂OH+(CF₃SO₂)₂O+C₅H₅N→BrCH₂CH₂CH₂OSO₂CF₃+C₅H₅NH⁺⁻OSO₂CF₃

10.89 g (38.6 mmol) of trifluoromethanesulfonic anhydride are slowly added with stirring at room temperature to a solution of 3.10 g (39.2 mmol) of dry pyridine in 40 ml of dry dichloromethane in a 250 ml flask with reflux condenser. The suspension is stirred for a further 30 min, and 5.20 g (37.4 mmol) of 3-bromopropanol are then added. After the reaction mixture has been stirred at room temperature for 1 hour, it is cooled to 0° C. The salt, pyridinium triflate, is filtered off and washed with 10 ml of cold (0° C.) and dry dichloromethane. The dichloromethane solutions are combined, and CH₂Cl₂ is distilled off in vacuo. The residue is subjected to fractional distillation after further filtration. 8.78 g of liquid 3-bromopropyl triflate are obtained. The yield of 3-bromopropyl triflate is 87%, based on 3-bromopropanol. The product, 3-bromopropyl triflate, is characterised by means of ¹H- and ¹⁹F-NMR spectra. The NMR data for the product, 3-bromopropyl triflate, are identical to the data described in Example 7a).

### 7c 1-Bromo-3- trifluoromethoxy)propane, BrCH₂CH₂CH₂OCF₃

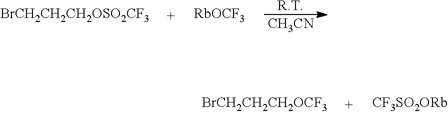

8.3 g (37.8 mmol) of trifluoromethyl triflate, CF₃SO₂OCF₃, are slowly added with stirring and cooling of the reaction mixture using a bath (0° C.) to 4.0 g (38.2 mmol) of dry rubidium fluoride suspended in 30 ml of dry acetonitrile in a 250 ml round-bottomed flask with reflux condenser which is cooled to −78° C. The reaction mixture is stirred at 20° C. for 2.5 hours. The temperature of the reflux condenser is then increased to room temperature in order to remove the trifluoromethylsulfonyl fluoride, CF₃SO₂F, formed during the reaction over the course of 30 min. 30 ml of dry acetonitrile and 8.2 g (30.3 mmol) of 3-bromopropyl triflate from Example 7a) are added at a bath temperature of 0° C. to the suspension of RbOCF₃ remaining in the flask, and the reaction mixture is stirred at room temperature for 60 hours. All volatile products are then distilled off in vacuo at 10⁻¹ mbar and room temperature into a cooled distillation trap (−196° C.). Fractional distillation gives 2.6 g of 1-bromo-3-(trifluoromethoxy)propane. The boiling point is 111-113° C. The yield of 1-bromo-3-(trifluoromethoxy)propane is 42%, based on 3-bromopropyl triflate.

The product, 1-bromo-3-(trifluoromethoxy)propane, is characterised by means of ¹H- and ¹⁹F-NMR spectra.

¹H-NMR (solvent: CDCl₃; reference substance: TMS), δ, ppm: 2.20 m (CH₂), 3.48 t (CH₂), 4.11 t (CH₂); ³J_{H,H}=5.9 Hz, ³J_{H,H}=6.3 Hz. ¹⁹F-NMR (solvent: CDCl₃; reference substance: CCl₃F), δ, ppm: −61.4 s (OCF₃).

### 7d) 1-Bromo-3-methylsulfonylpropane (3-bromopropyl mesylate)

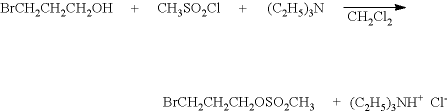

16.15 g (159.6 mmol) of triethylamine and 14.58 g (104.9 mmol) of 1-bromopropan-3-ol are dissolved in 150 ml of dry dichloromethane. 14.46 g (126.2 mmol) of methanesulfonyl chloride are added dropwise with stirring to the cold solution (bath temperature: 0° C.). The reaction mixture is stirred at 0° C. for 1 hour and at room temperature for 14 hours and then filtered. The solid is rinsed once with 25 ml and twice with 10 ml of dichloromethane. The combined filtrates are washed with 150 ml of water, 150 ml of 10% hydrochloric acid, with 150 ml of saturated sodium hydrogen-carbonate solution and 150 ml of saturated sodium chloride solution. The organic solution is dried using MgSO₄, filtered, and dichloromethane is evaporated. 22.2 g of crude product are obtained. The yield of 1-bromopropyl 3-mesylate is 97%, based on 3-bromopropanol. The crude product obtained can be subjected to fractional distillation in vacuo at 1 mbar (boiling point: 106-107.5° C./1 mbar). The product, 3-bromopropyl mesylate, is characterised by means of the ¹H-NMR spectrum.

¹H-NMR (solvent: CDCl₃; reference substance: TMS), δ, ppm: 3.02 s (CH₃), 2.26 m (CH₂); ³J_{H,H}=6.0 Hz, 3.50 t (CH₂); ³J_{H,H}=6.2 Hz, 4.37 t (CH₂); ³J_{H,H}=5.8 Hz.

This spectrum is identical to the spectrum of 3-bromopropyl mesylate described in the literature (W. K. Anderson et al., J. Med. Chem., Vol. 36, 1993, pp. 3618-3627).

### 7e) 1-Bromo-3- trifluoromethoxy)propane BrCH₂CH₂CH₂OCF₃

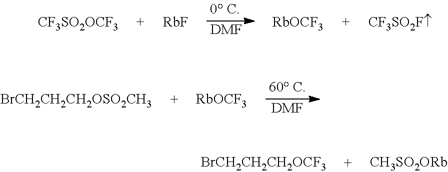

5.16 g (23.7 mmol) of trifluoromethyl triflate, CF₃SO₂OCF₃, are slowly added with stirring and cooling of the reaction mixture using a bath (0° C.) to 2.35 g (22.5 mmol) of dry rubidium fluoride suspended in 15 ml of dry N,N-dimethylformamide in a 100 ml round-bottomed flask with reflux condenser which is cooled to −78° C. The reaction mixture is stirred at 0° C. for 1 hour. The temperature of the reflux condenser is then increased to room temperature in order to remove the trifluoromethylsulfonyl fluoride, CF₃SO₂F, formed during the reaction over the course of 30 min. 5 ml of dry dimethylformamide and 3.73 g (17.2 mmol) of 3-bromopropyl mesylate from Example 7d) are added at a bath temperature of 0° C. to the suspension of RbOCF₃ remaining in the flask, and the reaction mixture is stirred at 50° C. for 15 hours and at 60° C. for 23 hours. All volatile products are then distilled off in vacuo at 10⁻¹ mbar and 50° C. into two distillation traps connected in series (−20° C. and −196° C.). 1.69 g of a liquid material which comprises 1-bromo-3-(trifluoromethoxy)propane (25%), 1,3-bis(trifluoromethoxy)propane (27%), 1,3-dibromopropane (2%) and DMF (46%), are obtained in the rear cold trap.

The yield of 1-bromo-3-(trifluoromethoxy)propane is 12%, based on 1-bromopropyl 3-mesylate.

The product, 1-bromo-3-(trifluoromethoxy)propane, is characterised by means of ¹H- and ¹⁹F-NMR spectra. The NMR data for the product, 1-bromo-3-(trifluoromethoxy)propane, are identical to the data described in Example 7c).

### Example 8

### 1-(Trifluoromethoxy)hex-5-ene, CH₂═CH(CH₂)₄OCF₃

### 8a) 1-Trifluoromethylsulfonylhex-5-ene (5-hexen-1-yl triflate)

CH₂═CH(CH₂)₃CH₂OH+(CF₃SO₂)₂O+C₅H₅N→CH₂═CH(CH₂)₃CH₂OSO₂CF₃+C₅H₅NH⁺⁻OSO₂CF₃

162.3 g (575 mmol) of trifluoromethanesulfonic anhydride are slowly added with stirring at room temperature to the solution of 47.8 g (604 mmol) of dry pyridine in 1.6 l of dry dichloromethane in a 2 litre flask with reflux condenser. The suspension is stirred for a further 30 min, cooled to −20° C., and 56.6 g (565 mmol) of hex-5-en-1-ol are then added. After the reaction mixture has been stirred at room temperature for 1 hour, it is cooled to −78° C. The salt, pyridinium triflate, is filtered off and washed twice with 50 ml of cold (−78° C.) and dry dichloromethane. The dichloromethane solutions are combined, and CH₂Cl₂ is distilled off in vacuo at 0° C. The suspension now obtained is cooled to −20° C. and re-filtered. The solid is rinsed twice with 20 ml of cold (−20° C.) and dry n-pentane. The pentane is removed in vacuo at 0° C. The substance obtained in this way, hex-5-enyl triflate (116 g), is not purified further, but instead stored at −78° C. and reacted further as crude product (Example 8b). The yield of hex-5-enyl triflate is 88.5%, based on hex-5-en-1-ol. The product, hex-5-enyl triflate, is characterised by means of the ¹H-NMR spectrum.

¹H-NMR (without solvent, reference substance: TMS), δ, ppm: 1.48 m (CH₂), 1.79 m (CH₂), 2.07 m (CH₂), 4.51 t (CH₂), 4.96 d,m (2H), 5.74 d,d,t (1H); ³J_{H,H}=6.8 Hz, ³J_{H,H}=10.3 Hz, ³J_{H,H}=17.1 Hz. ¹⁹F-NMR (solvent: CD₃CN; reference substance: CCl₃F), δ, ppm: −77.7 s (CF₃).

### 8b) 1-(Trifluoromethoxy)hex-5-ene, CH₂═CH(CH₂)₄OCF₃

CH₂═CH(CH₂)₄OSO₂CF₃+RbOCF₃→CH₂═CH(CH₂)₄OCF₃+RbOSO₂CF₃

143.6 g (658.5 mmol) of trifluoromethyl triflate, CF₃SO₂OCF₃, are slowly added with stirring and cooling of the reaction mixture using a bath (0° C.) to 63.0 g (603 mmol) of dry rubidium fluoride suspended in 300 ml of dry acetonitrile in a 1 l round-bottomed flask with reflux condenser which is cooled to −78° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 300 ml of dry acetonitrile and 104.5 g (450 mmol) of hex-5-enyl triflate from Example 8a) are added at a bath temperature of 0° C. to the suspension of RbOCF₃ remaining in the flask, and the reaction mixture is stirred at room temperature for 63 hours. All volatile products are then condensed off in vacuo at 10⁻¹ mbar and a bath temperature of 30° C. into a cooled distillation trap (−196° C.). 100 ml of dry n-pentane are subsequently added to the products condensed off, and the acetonitrile is removed by azeotropic distillation. The residue is subjected to fractional re-condensation at 0.4 mbar into two traps (at −40° C. and −196° C.). After distillation of the liquid from the first trap, 32 g of 1-trifluoromethoxyhex-5-ene are obtained (boiling point: 111-112° C.). The yield of 1-trifluoromethoxyhex-5-ene is 43%, based on hex-5-enyl triflate. The product, 1-trifluoromethoxyhex-5-ene, is characterised by means of ¹H- and ¹⁹F-NMR spectra.

¹H-NMR (solvent: CDCl₃; reference substance: TMS), δ, ppm: 1.48 m (CH₂), 1.69 m (CH₂), 2.07 m (CH₂), 3.94 t (CH₂), 4.96 d,m (1 H; cis-H₂C═CH—), 5.01 d,m (1 H; trans-H₂C═CH—), 5.77 d,d,t (1H); ³J_{H,H}=6.5 Hz, ³J_{H,H}=6.7 Hz, ³J_{H,H}=10.3 Hz, ³J_{H,H}=17.1 Hz. ¹⁹F-NMR (solvent: CDCl₃; reference substance: CCl₃F), δ, ppm: −60.5 s (OCF₃).

### 8c) 1-Methylsulfonylhex-5-ene (1-mesylhex-5-ene)

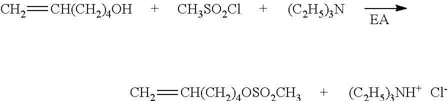

86.1 g (752 mmol) of methanesulfonyl chloride are slowly added with stirring to a cold (−10° C.) solution of 62.0 g (619 mmol) of hex-5-en-1-ol and 101.44 g (1003 mmol) of triethylamine in 1000 ml of ethyl acetate in a 2 litre flask at such a rate that the internal temperature remains below 5° C. The suspension is stirred at room temperature for 12 hours. The salt, triethylammonium chloride, is filtered off and washed a number of times with ethyl acetate. The combined filtrates are evaporated to about 600 ml and washed with 75 ml of 10% hydrochloric acid, with 50 ml of saturated sodium hydrogencarbonate solution and with 50 ml of saturated sodium chloride solution. The organic phase is freed from solvent in a rotary evaporator, taken up in 500 ml of dichloromethane and dried using MgSO₄. The mixture is then filtered, dichloromethane is evaporated, and the residue is subjected to fractional distillation in vacuo (boiling point: 80° C./3·10⁻³ mbar). 101.2 g of liquid 1-mesylhex-5-ene are obtained. The yield of 1-mesylhex-5-ene is 92%, based on hex-5-en-1-ol. The product, 1-mesylhex-5-ene, is characterised by means of the ¹H-NMR spectrum.

¹H-NMR (CD₃CN, reference substance: TMS), δ, ppm: 1.43-1.53 m (CH₂), 1.67-1.77 m (CH₂), 2.05-2.13 q (CH₂), 3.00 s (CH₃), 4.20 t (CH₂); ³J_{H,H}=6.5 Hz, 4.97 d,m (1H; cis-H, H₂C═CH—), 5.04 d,m (1 H; trans-H, H₂C═CH—), 5.84 d,d,t (1H); ³J_{H,H}=6.8 Hz, ³J_{H,H}=10.3 Hz, ³J_{H,H}=17.0 Hz.

This spectrum is identical to the spectrum of 1-mesylhex-5-ene described in the literature (O. Phanstiel et al., J. Med. Chem., Vol. 48, 2005, pp. 3832-3839).

### 8d) 1-(Trifluoromethoxy)hex-5-ene, CH₂═CH(CH₂)₄OCF₃

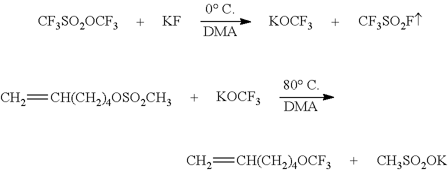

175.3 g (804.0 mmol) of trifluoromethyl triflate, CF₃SO₂OCF₃, are slowly added with stirring and cooling of the reaction mixture using a bath (0° C.) to 43.4 g (746 mmol) of dry potassium fluoride suspended in 870 ml of dry N,N-dimethylacetamide in a 2 l round-bottomed flask with reflux condenser which is cooled to −78° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 101.0 g (567 mmol) of hex-5-enyl mesylate from Example 8c) are added to the suspension of KOCF₃ remaining in the flask at a bath temperature of 0° C., and the reaction mixture is stirred at 80° C. for 48 hours. The reaction mixture is then cooled to room temperature, 300 ml of n-pentane are added with stirring, and the mixture is filtered. The salt is washed twice with 300 ml of n-pentane, and the combined organic phases are washed a number of times with water and dried using MgSO₄. After filtration, the pentane is distilled off in vacuo, and the residue is subjected to fractional distillation (boiling point: 111-112° C.). 62.9 g of liquid 1-trifluoromethoxyhex-5-ene are obtained. The yield of 1-trifluoromethoxyhex-5-ene is 66%, based on hex-5-enyl mesylate. The product, 1-trifluoromethoxyhex-5-ene, is characterised by means of ¹H- and ¹⁹F-NMR spectra. The NMR data for the product, 1-trifluoromethoxyhex-5-ene, are identical to the data in Example 8b).

### Example 9

### 1-(Trifluoromethoxy)dec-9-ene, CH₂═CH(CH₂)₇CH₂OCF₃

### 9a) 1-Trifluoromethylsulfonyldec-9-ene (dec-9-enyl triflate)

CH₂═CH(CH₂)₇CH₂OH+(CF₃SO₂)₂O+C₅H₅N→CH₂═CH(CH₂)₇CH₂OSO₂CF₃+C₅H₅NH⁺⁻OSO₂CF₃

53.7 g (190 mmol) of trifluoromethanesulfonic anhydride are slowly added with stirring at room temperature to the solution of 16.5 g (209 mmol) of dry pyridine in 380 ml of dry dichloromethane in a 1 litre flask with reflux condenser. The suspension formed is stirred for a further 30 min, cooled to −20° C., and 26.7 g (171 mmol) of dec-9-en-1-ol are then added. After the reaction mixture has been stirred at 0° C. (bath temperature) for 1.5 hours, it is cooled to −78° C. The salt, pyridinium triflate, is filtered off and washed twice with 50 ml of cold (−78° C.) and dry dichloromethane. The dichloromethane solutions are combined, and CH₂Cl₂ is distilled off in vacuo at 0° C. The suspension now obtained is cooled to −20° C., diluted with 50 ml of dry n-pentane and re-filtered. The solid is rinsed twice with 20 ml of cold (−20° C.) and dry n-pentane. The pentane is removed in vacuo at 0° C. The substance obtained in this way, dec-9-enyl triflate (43.3 g), is not purified further, but instead is stored at −78° C. and reacted further as crude product (Example 9b). The yield of dec-9-enyl triflate is 88%, based on dec-9-en-1-ol. The product, dec-9-enyl triflate, is characterised by means of ¹H- and ¹⁹F-NMR spectra.

¹H-NMR (solvent: CDCl₃; reference substance: TMS), δ, ppm: 1.20-1.44 m (10H, 5CH₂), 1.80 quin (CH₂), 2.02 m (CH₂), 4.51 t (CH₂), 4.91 d,m (1H; cis-H₂C═CH—), 4.97 d,m (1H; trans-H₂C═CH—), 5.78 d,d,t (1H); ³J_{H,H}=6.6 Hz, ³J_{H,H}=6.7 Hz, ³J_{H,H}=10.1 Hz, ³J_{H,H}=17.0 Hz. ¹⁹F-_{NMR} (_{solvent: CDCl}₃; reference substance: CCl₃F), δ, ppm: −75.4 s (CF₃).

### 9b) 1-(Trifluoromethoxy)dec-9-ene CH₂═CH(CH₂)₇CH₂OCF₃

CH₂═CH(CH₂)₈OSO₂CF₃+RbOCF₃→CH₂═CH(CH₂)₈OCF₃+RbOSO₂CF₃

20.82 g (95.5 mmol) of trifluoromethyl triflate, CF₃SO₂OCF₃, are slowly added with stirring and cooling of the reaction mixture using a bath (0° C.) to 9.32 g (89.2 mmol) of dry rubidium fluoride suspended in 45 ml of dry acetonitrile in a 250 ml round-bottomed flask with reflux condenser which is cooled to −78° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 45 ml of dry acetonitrile and 20.5 g (71.0 mmol) of dec-9-enyl triflate from Example 9a) are added to the suspension of RbOCF₃ remaining in the flask at a bath temperature of 0° C., and the reaction mixture is stirred firstly at 15° C. for 25 hours and then at room temperature for 90 hours. Acetonitrile is condensed off in a membrane-pump vacuum at room temperature into a cold trap (−196° C.). The residue is subsequently condensed off in vacuo at 10⁻¹ mbar (oil pump) and a bath temperature of 50° C. into a further cooled distillation trap (−196° C.). The collected liquid product is distilled in vacuo at 1.5 mbar (boiling point: 38° C.). 6.3 g of liquid 1-trifluoromethoxydec-9-ene are obtained. The yield of 1-trifluoromethoxydec-9-ene is 40%, based on dec-9-enyl triflate. The product, 1-trifluoromethoxydec-9-ene, is characterised by means of ¹H- and ¹⁹F-NMR spectra.

¹H-NMR (solvent: CDCl₃; reference substance: TMS), δ, ppm: 1.23-1.43 m (10H, 5CH₂), 1.66 quin (CH₂), 2.02 q (CH₂), 3.93 t (CH₂), 4.91 d,m (1H; cis-H₂C═CH—), 4.98 d,m (1H; trans-H₂C═CH—), 5.79 d,d,t (1H); ³J_{H,H}=6.5 Hz, ³J_{H,H}=6.7 Hz, ³J_{H,H}=10.3 Hz, ³J_{H,H}=17.0 Hz. ¹⁹F-NMR (solvent: CDCl₃; reference substance: CCl₃F), δ, ppm: −61.4 s (OCF₃).

### Example 10

### 1-(Trifluoromethoxy)dec-9-ene, CH₂═CH(CH₂)₇CH₂OCF₃

### 10a) 1-Methylsulfonyldec-9-ene (9-decen-1-yl mesylate)

CH₂═CH(CH₂)₇CH₂OH+CH₃SO₂Cl+(C₂H₅)₃N→CH₂═CH(CH₂)₇CH₂OSO₂CH₃+(C₂H₅)₃NH⁺Cl⁻

76.9 g (760.1 mmol) of triethylamine and 73.7 g (471.5 mmol) of dec-9-en-1-ol are dissolved in 600 ml of ethyl acetate. 67.2 g (586.5 mmol) of methanesulfonyl chloride are added dropwise with stirring to the cold solution (bath temperature: 0° C.). The reaction mixture is stirred at room temperature for 1 hour and then filtered. The solid is rinsed five times with 100 ml of ethyl acetate. The combined filtrates are evaporated to about 500 ml and washed with 50 ml of 10% hydrochloric acid, 50 ml of saturated sodium hydrogencarbonate solution and 50 ml of saturated sodium chloride solution. The solvents are removed with the aid of a rotary evaporator. The residue is dissolved in 200 ml of dichloromethane and dried using MgSO₄. The solution is filtered, and dichloromethane is evaporated. The crude product obtained is subjected to fractional distillation in vacuo at 0.03 mbar. 97.7 g of dec-9-enyl mesylate are obtained (boiling point: 103° C./0.03 mbar). The yield of dec-9-enyl mesylate is 88%, based on dec-9-en-1-ol. The product, dec-9-enyl mesylate, is characterised by means of the ¹H-NMR spectrum. ¹H-NMR (solvent: CDCl₃; reference substance: TMS), δ, ppm: 1.22-1.46 m (10H, 5CH₂), 1.72 quin (CH₂), 2.03 m (CH₂), 2.97 s (CH₃), 4.19 t (CH₂), 4.90 d,m (1H; cis-H₂C═CH—), 4.97 d,m (1H; trans-H₂C═CH—), 5.78 d,d,t (1H); ³J_{H,H}=6.5 Hz, ³J_{H,H}=6.8 Hz, ³J_{H,H}=10.3 Hz, ³J_{H,H}=17.0 Hz. This spectrum is identical to the spectrum of 1-methylsulfonyldec-9-ene described in the literature (J. Morita et al., Green Chem., Vol. 7, 2005, pp. 711-715).

### 10b) 1-Trifluoromethoxy)dec-9-ene CH₂═CH(CH₂)₇CH₂OCF₃

CH₂═CH(CH₂)₈OSO₂CH₃+RbOCF₃→CH₂═CH(CH₂)₈OCF₃+RbOSO₂CH₃

27.3 g (125.1 mmol) of trifluoromethyl triflate, CF₃SO₂OCF₃, are slowly added with stirring and cooling of the reaction mixture using a bath (0° C.) to 12.4 g (118.6 mmol) of dry rubidium fluoride suspended in 130 ml of dry N,N-dimethylformamide in a 0.5 l round-bottomed flask with reflux condenser which is cooled to −78° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 130 ml of dry N,N-dimethylformamide and 21.9 g (93.6 mmol) of dec-9-enyl mesylate from Example 8a) are added to the suspension of RbOCF₃ remaining in the flask at a bath temperature of 0° C., and the reaction mixture is stirred at 80° C. for 72 hours. All volatile products are then condensed off in vacuo at 0.02 mbar and a max. bath temperature of 50° C. into a cooled distillation trap (−196° C.). The liquid obtained is poured into 800 ml of ice-water and extracted twice with 200 ml of n-pentane. The organic phase is separated off, dried using MgSO₄, and n-pentane is removed in a rotary evaporator. The crude product obtained, 1-trifluoromethoxydec-9-ene (14.5 g; yield: 69%), is distilled in vacuo at 20 mbar (boiling point: 76° C./20 mbar, 38° C./1.5 mbar). The product, 1-trifluoromethoxydec-9-ene, is characterised by means of ¹H- and ¹⁹F-NMR spectra.

¹H-NMR (solvent: CDCl₃; reference substance: TMS), δ, ppm: 1.23-1.43 m (10H, 5CH₂), 1.66 quin (CH₂), 2.02 q (CH₂), 3.93 t (CH₂); ³J_{H,H}=6.5 Hz, 4.91 d,m (1H; cis-H₂C═CH—); ³J_{H,H}=10.3 Hz, 4.98 d,m (1H; trans-H₂C═CH—); ³J_{H,H}=17.0 Hz, 5.79 d,d,t (1H); ³J_{H,H}=6.7 Hz, ³J_{H,H}=10.3 Hz, ³J_{H,H}=17.0 Hz. ¹⁹F-NMR (solvent: CDCl₃; reference substance: CCl₃F), δ, ppm: −61.4 s (OCF₃).

### Example 11

### 1-(Trifluoromethoxy)butane, n-C₄H₉OCF₃

n-C₄H₉OSO₂OC₄H₉-n+RbOCF₃→n-C₄H₉OCF₃+RbOSO₂C₄H₉

6.45 g (29.6 mmol) of trifluoromethyl triflate, CF₃SO₂OCF₃, are slowly added with stirring and cooling of the reaction mixture using a bath (0° C.) to 2.75 g (26.3 mmol) of dry rubidium fluoride suspended in 20 ml of dry N,N-dimethylformamide in a 100 ml round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 15 ml of dry N,N-dimethylformamide and 4.37 g (20.8 mmol) of di-n-butyl sulfate are added to the suspension of RbOCF₃ remaining in the flask at a bath temperature of 0° C., and the reaction mixture is stirred at 70° C. for 72 hours. All volatile products are then condensed off in vacuo at 20 mbar and a max. bath temperature of 50° C. into a cooled distillation trap (−196° C.). The solid substance obtained is slowly warmed to room temperature, and 1-(trifluoromethoxy)butane is collected in a cooled trap (0° C.). 0.36 g of liquid 1-(trifluoromethoxy)butane is obtained. The yield of 1-(trifluoromethoxy)butane is 12.3%, based on dibutyl sulfate. The product, 1-(trifluoromethoxy)butane, is characterised by means of ¹H- and ¹⁹F-NMR spectra.

¹H-NMR (solvent: CDCl₃; reference substance: TMS), δ, ppm: 0.91 t (CH₃); ³J_{H,H}=7.3 Hz, 1.39 m (CH₂), 1.64 m (CH₂), 3.93 t (CH₂); ³J_{H,H}=6.4 Hz. ¹⁹F-NMR (solvent: CDCl₃; reference substance: CCl₃F), δ, ppm: −60.5 s (OCF₃).

### Example 12

### α-(Trifluoromethoxy)ethyl acetate, CF₃OCH₂C(O)OC₂H₅

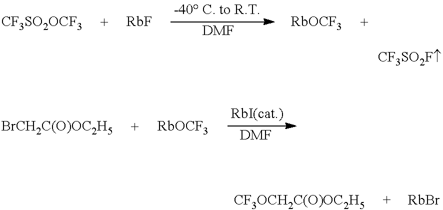

9.31 g (89.2 mmol) of dry rubidium fluoride are suspended in 100 ml of dry dimethylformamide in a 250 ml round-bottomed flask with reflux condenser (cooled to −40° C.), and 21.4 g (98.2 mmol) of trifluoromethyl triflate, CF₃SO₂OCF₃, are slowly added with stirring and cooling of the reaction mixture by means of a bath cooled to −40° C. The reaction mixture is kept at −20° C. for 30 min, at 0° C. for 1 hour and at 20° C. for 30 min. The temperature of the reflux condenser is then increased to −20° C. in order to remove the trifluoromethylsulfonyl fluoride, CF₃SO₂F, which is formed during the reaction. 2.1 g (9.9 mmol) of rubidium iodide and 13.25 g (200 mmol) of α-bromoethyl acetate, BrCH₂C(O)OC₂H₅, are added to the suspension of RbOCF₃ which remains, and the reaction mixture is stirred at 60° C. for 48 hours. All volatile products are then distilled off in vacuo at 10⁻³ mbar and 50° C. into a cooled distillation trap (−196° C.). In total, 64.9 g of a liquid material are obtained which comprises CF₃OCH₂C(O)OC₂H₅ (9.4%), FCH₂C(O)OC₂H₅ (2.0%), BrCH₂C(O)OC₂H₅ (2.3%) and DMF (86.3%). This mixture is subjected to fractional distillation, and α-(trifluoromethoxy)ethyl acetate is isolated and characterised by spectroscopy.

NMR data (0° C.): ¹H-NMR (solvent: CD₃CN; reference substance: TMS), δ, ppm: 1.12 t (CH₃), 4.10 q (CH₂), 4.71 s (CH₂); ³J_{H,H}=7.1 Hz. ¹⁹F-NMR (solvent: CD₃CN; reference substance: CCl₃F), δ, ppm: −59.3 s (OCF₃).

### Example 13a

### Ethyl 2-methylsulfonylacetate (ethyl 2-mesylacetate), CH₃SO₂OCH₂C(O)OC₂H₅

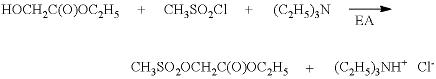

112.6 g (1112.8 mmol) of triethylamine and 76.4 g (734.1 mmol) of ethyl 2-hydroxyacetate (glycolic acid ethyl ester) are dissolved in 600 ml of ethyl acetate. 102.9 g (898.1 mmol) of methanesulfonyl chloride are added drop-wise with stirring to the cold solution (bath temperature: 0° C.). The reaction mixture is stirred at room temperature for 1 hour and then filtered. The solid is rinsed once with 100 ml and twice with 200 ml of ethyl acetate. The combined filtrates are washed with 200 ml of water, twice with 100 ml of 10% hydrochloric acid, 100 ml of saturated sodium hydrogencarbonate solution and 100 ml of saturated sodium chloride solution. The solvents are removed with the aid of a rotary evaporator. The residue is dissolved in 100 ml of dichloromethane and dried using MgSO₄. The solution is filtered, and dichloromethane is evaporated. The crude product obtained is subjected to fractional distillation in vacuo at 0.008 mbar. 127.6 g of ethyl 2-mesylacetate are obtained (boiling point: 103° C./0.03 mbar). The yield of ethyl 2-mesylacetate is 95%, based on ethyl 2-hydroxyacetate. The product, ethyl 2-mesylacetate, is characterised by means of the ¹H-NMR spectrum.

¹H-NMR (solvent: CDCl₃; reference substance: TMS), δ, ppm: 1.28 t (CH₃); ³J_{H,H}=7.2 Hz, 3.18 s (SO₂CH₃), 4.24 q (CH₂); ³J_{H,H}=7.2 Hz, 4.73 s (CH₂).

### Example 13b

### α-(Trifluoromethoxy)ethyl acetate (ethyl 2-(trifluoromethoxy)acetate), CF₃OCH₂C(O)OC₂H₅

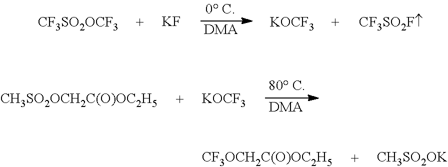

26.6 g (458.0 mmol) of dry potassium fluoride are suspended in 350 ml of dry N,N-dimethylacetamide (DMA) in a 500 ml round-bottomed flask with reflux condenser (cooled to −78° C.) and cooled to 0° C. 107.7 g (493.6 mmol) of trifluoromethyl triflate, CF₃SO₂OCF₃, are slowly added with stirring and cooling of the reaction mixture by means of a bath cooled to 0° C. The reaction mixture is kept at 0° C. for 1 hour. The temperature of the reflux condenser is then increased to room temperature in order to remove the trifluoromethylsulfonyl fluoride, CF₃SO₂F, which is formed during the reaction. 65.9 g (361.6 mmol) of ethyl 2-mesylacetate, CH₃SO₂OCH₂C(O)OC₂H₅, are added to the suspension of KOCF₃ which remains, and the reaction mixture is stirred at 75-80° C. for 17 hours. Then reaction mixture is then cooled by means of an ice bath (0° C.), and 160 ml of water are added. The organic phase is separated off, and the aqueous solution is extracted three times with n-pentane. The organic phases are combined, dried using MgSO₄, filtered, and the solvent is evaporated. The residue is subjected to fractional distillation (boiling point: 121-122° C.). 42.1 g of liquid ethyl 2-(trifluoromethoxy)acetate are obtained. The yield of ethyl 2-(trifluoromethoxy)acetate is 68%, based on ethyl 2-mesylacetate. The product, ethyl 2-(trifluoromethoxy)acetate, is characterised by means of ¹H- and ¹⁹F-NMR spectra. The NMR data for the product, ethyl 2-(trifluoromethoxy)acetate, are identical to the data in Example 12.

### Example 14

### α-(Trifluoromethoxy)ethyl acetate (ethyl 2-(trifluoromethoxy)acetate), CF₃OCH₂C(O)OC₂H₅

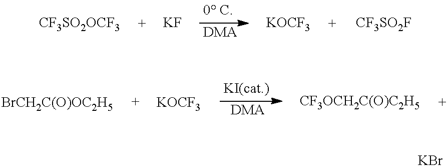

31.3 g (539.5 mmol) of dry potassium fluoride are suspended in 140 ml of dry N,N-dimethylacetamide (DMA) in a 500 ml round-bottomed flask with reflux condenser (cooled to −78° C.) and cooled to 0° C. 125.7 g (576.6 mmol) of trifluoromethyl triflate, CF₃SO₂OCF₃, are slowly added with stirring and cooling of the reaction mixture by means of a bath cooled to 0° C. The reaction mixture is kept at 0° C. for 1 hour. The temperature of the reflux condenser is then increased to room temperature in order to remove the trifluoromethylsulfonyl fluoride, CF₃SO₂F, which is formed during the reaction. 70.9 g (424.3 mmol) of ethyl 2-bromoacetate, BrCH₂C(O)OC₂H₅, and 7.0 g (4.2 mmol) of potassium iodide are added to the suspension of KOCF₃ which remains, and the reaction mixture is stirred at 50° C. for 49 hours. The reaction mixture is then cooled by means of an ice bath (0° C.), and 200 ml of water are added. The organic phase is separated off, and the aqueous solution is extracted three times with n-pentane. The organic phases are combined and washed twice with water, once with saturated sodium hydrogencarbonate solution, once with water and once with sodium thiosulfate solution. The solution is dried using MgSO₄, filtered, and the solvent is evaporated. The residue is subjected to fractional distillation (boiling point: 121-122° C.). 54.0 g of liquid ethyl 2-(trifluoromethoxy)acetate are obtained. The yield of ethyl 2-(trifluoromethoxy)acetate is 74%, based on ethyl 2-bromoacetate. The product, ethyl 2-(trifluoromethoxy)acetate, is characterised by means of ¹H- and ¹⁹F-NMR spectra. The NMR data for the product, ethyl 2-(trifluoromethoxy)acetate, are identical to the data in Example 12.

### Example 15

### 1-Chloro-3-(trifluoromethoxy)propane, ClCH₂CH₂CH₂OCF₃

ClCH₂CH₂CH₂OSO₂Cl+RbOCF₃→ClCH₂CH₂CH₂OCF₃+RbOSO₂Cl

3.89 g (17.8 mmol) of trifluoromethyl triflate, CF₃SO₂OCF₃, are slowly added with stirring and cooling of the reaction mixture using a bath (0° C.) to 2.08 g (19.9 mmol) of dry rubidium fluoride suspended in 24 ml of dry acetonitrile in a 100 ml round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 24 ml of dry acetonitrile and 2.52 g (13.1 mmol) of 3-chloropropylsulfonyl chloride are added to the suspension of RbOCF₃ remaining in the flask at a bath temperature of 0° C., and the reaction mixture is stirred at room temperature for 48 hours. All volatile products are then condensed off in vacuo at 0.7 mbar and a max. bath temperature of 50° C. into a cooled distillation trap (−196° C.). 38.7 g of products which are liquid at room temperature are obtained. According to the ¹H-NMR spectrum, the mixture comprises 96.7% of acetonitrile, 1.7% of 1-chloro-3-(trifluoromethoxy)propane and 1.6% of 1,3-dichloropropane. The yield of 1-chloro-3-(trifluoromethoxy)propane is 30% (NMR), based on 3-chloropropylsulfonyl chloride. The 1-chloro-3-(trifluoromethoxy)propane can be isolated from the mixture by distillation. NMR data for 1-chloro-3-(trifluoromethoxy)propane:

¹H-NMR (solvent: CDCl₃; reference substance: TMS), δ, ppm: 2.02 m (CH₂), 3.53 t (CH₂), 4.02 t (CH₂); ³J_{H,H}=5.9 Hz, ³J_{H,H}=6.2 Hz. ¹⁹F-NMR (solvent: CDCl₃; reference substance: CCl₃F), δ, ppm: −60.7 s (OCF₃).

### Example 16

### 1-(Trifluoromethoxy)hex-5-ene, CH₂═CH(CH₂)₄OCF₃

### 16a) 1-Trifluoroacetylhex-5-ene (5-hexen-1-yl trifluoroacetate), CH₂═CH(CH₂)₄OC(O)CF₃

CH₂═CH(CH₂)₃CH₂OH+(CF₃CO)₂O+C₅H₅N→CH₂═CH(CH₂)₃CH₂OC(O)CF₃+C₅H₅NH⁺⁻OC(O)CF₃

50.7 g (241 mmol) of trifluoroacetic anhydride are slowly added with stirring at 0° C. (ice bath) to the solution of 19.3 g (244 mmol) of dry pyridine and 23.0 g (230 mmol) of hex-5-en-1-ol in 150 ml of dry diethyl ether. The suspension is warmed to room temperature and stirred for a further 12 hours. The salt, pyridinium trifluoroacetate, is filtered off and washed twice with 50 ml of dry diethyl ether. The diethyl ether solutions are combined, and (C₂H₅)₂O is distilled off in vacuo at room temperature. The suspension now obtained is re-filtered, and the solid is washed three times with 10 ml of n-pentane. The filtrates are combined, and pentane is removed in vacuo. The residue is subjected to fractional distillation in vacuo at 50 mbar. 32.3 g of liquid substance (boiling point: 70° C./50 mbar) are obtained. The yield of 5-ₕₑₓₑₙ₋1-yl trifluoroacetate is 72%, based on hex-5-en-1-ol. The product, 5-hexen-1-yl trifluoroacetate, is characterised by means of ¹H- and ¹⁹F-NMR spectra.

¹H-NMR (solvent: CDCl₃; reference substance: TMS), δ, ppm: 1.47 quin (CH₂), 1.75 quin (CH₂), 2.08 q (CH₂), 4.34 t (CH₂), 4.97 d,m (1 H; cis-H₂C═CH—), 5.01 d,m (1H; trans-H₂C═CH—), 5.76 d,d,t (1H); ³J_{H,H}=6.6 Hz, ³J_{H,H}=6.7 Hz, ³J_{H,H}=10.3 Hz, ³J_{H,H}=17.0 Hz. ¹⁹F-NMR (solvent: CDCl₃; reference substance: CCl₃F), δ, ppm: −74.9 s (CF₃).

### 16b) 1-(Trifluoromethoxy)hex-5-ene, CH₂═CH(CH₂)₄OCF₃

CH₂═CH(CH₂)₄OC(O)CF₃+RbOCF₃→CH₂═CH(CH₂)₄OCF₃+RbOC(O)CF₃

27 g (124 mmol) of trifluoromethyl triflate, CF₃SO₂OCF₃, are slowly added with stirring and cooling of the reaction mixture using a bath (0° C.) to 12.55 g (120 mmol) of dry rubidium fluoride suspended in 54 ml of dry N,N-dimethylformamide in a 250 ml round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 26 ml of dry N,N-dimethylformamide and 18.2 g (92.8 mmol) of 5-hexen-1-yl trifluoroacetate from Example 12a) are added to the suspension of RbOCF₃ remaining in the flask at a bath temperature of 0° C., and the reaction mixture is stirred at 100° C. to 120° C. for 124 hours. All volatile products are then condensed off in vacuo at 0.1 mbar and a max. bath temperature of 50° C. into a cooled distillation trap (−196° C.). A mixture of products which are liquid at room temperature is obtained. According to the ¹H-NMR spectrum, about 12% of starting material is converted into product, 1-trifluoromethoxyhex-5-ene. The NMR data for the product, 1-trifluoromethoxyhex-5-ene, are identical to the data in Example 8b).

### Example 17

### 1-(Trifluoromethoxy)dec-9-ene, CH₂═CH(CH₂)₇CH₂OCF₃

CH₂═CH(CH₂)₈OSO₂CH₃+RbOCF₃→CH₂═CH(CH₂)₈OCF₃+RbOSO₂CH₃

39.2 g (179.8 mmol) of trifluoromethyl triflate, CF₃SO₂OCF₃, are slowly added with stirring and cooling of the reaction mixture using a bath (0° C.) to 17.5 g (167.5 mmol) of dry rubidium fluoride suspended in 180 ml of dry N,N-dimethylacetamide in a 0.5 l round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 180 ml of dry N,N-dimethylacetamide and 30.6 g (130.6 mmol) of dec-9-enyl mesylate (from Example 8a) are added to the suspension of RbOCF₃ remaining in the flask at a bath temperature of 0° C., and the reaction mixture is stirred at 75-80° C. (bath temperature) for 50 hours. The solid substance precipitated is filtered off, the filtrate is poured into 800 ml of ice-water and extracted twice with 100 ml of n-pentane. The solid salt is mixed with 200 ml of water and extracted with n-pentane (2×100 ml). The extracts are combined and washed with water. After drying using MgSO₄, n-pentane is removed in a rotary evaporator. The residue is re-condensed in vacuo at 1×10⁻³ mbar and at 40° C. The crude product obtained (purity: 96%), 1-trifluoromethoxydec-9-ene (26.6 g; yield: 91%), is distilled in vacuo at 20 mbar (boiling point: 76° C./20 mbar, 38° C./1.5 mbar). The product, 1-trifluoromethoxydec-9-ene, is characterised by means of ¹H- and ¹⁹F-NMR spectra. The NMR data for the product, 1-trifluoromethoxydec-9-ene, are identical to the data in Example 10b).

### Example 18

### 1-(Trifluoromethoxy)dec-9-ene, CH₂═CH(CH₂)₇CH₂OCF₃

CH₂═CH(CH₂)₈OSO₂CH₃+KOCF₃→CH₂═CH(CH₂)₈OCF₃+KOSO₂CH₃

41.8 g (191.7 mmol) of trifluoromethyl triflate, CF₃SO₂OCF₃, are slowly added with stirring and cooling of the reaction mixture using a bath (0° C.) to 10.3 g (177.3 mmol) of dry potassium fluoride suspended in 190 ml of dry N,N-dimethylacetamide in a 0.5 l round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 190 ml of dry N,N-dimethylacetamide and 32.9 g (140.4 mmol) of dec-9-enyl mesylate (from Example 8a) are added to the suspension of KOCF₃ remaining in the flask at a bath temperature of 0° C., and the reaction mixture is stirred at 75-80° C. (bath temperature) for 48 hours. The solid substance precipitated is filtered off, the filtrate is poured into 800 ml of ice-water and extracted twice with 100 ml of n-pentane. The solid salt is extracted with n-pentane (3×100 ml). The extracts are combined and washed with water. After drying using MgSO₄, the drying agent is filtered off, and n-pentane is removed in a rotary evaporator. The residue is re-condensed in vacuo at 1×10⁻³ mbar and 40° C. 27.3 g of 1-trifluoromethoxydec-9-ene (purity: 98%; yield: 87%) are obtained. The product, 1-trifluoromethoxydec-9-ene, is characterised by means of ¹H- and ¹⁹F-NMR spectra. The NMR data for the product, 1-trifluoromethoxydec-9-ene, are identical to the data in Example 10b).

### Example 19

### 1,4-Bis(trifluoromethoxy)but-2-ene

### 19a) 2-Butenyl 1,4-dimesylate, CH₃SO₂OCH₂CH═CHCH₂OSO₂CH₃

HOCH₂CH═CHCH₂OH+2 CH₃SO₂Cl+2 (C₂H₅)₃N→→CH₃SO₂OCH₂CH═CHCH₂OSO₂CH₃+2 (C₂H₅)₃NH⁺Cl⁻

A mixture of 17.7 g (200.5 mmol) of cis-but-2-ene-1,4-diol and 45.1 g (445.9 mmol) of triethylamine in 330 ml of dichloromethane is slowly added dropwise with stirring to a cold solution (bath temperature: −10° C.) of 51.1 g (445.8 mmol) of methanesulfonyl chloride in 330 ml of dichloromethane. The reaction mixture is stirred at 0° C. for 1 hour, transferred into a separating funnel and washed by shaking three times with 70 ml of cold saturated NaHCO₃ solution. The organic phase obtained is dried using MgSO₄, filtered, and the solvents are removed with the aid of a rotary evaporator. The product (pale-yellow oil) slowly crystallises through on cooling. The yield of 2-butenyl 1,4-dimesylate is 48.5 g (99%), based on cis-but-2-ene-1,4-diol. The product, 2-butenyl 1,4-dimesylate, is characterised by means of the ¹H-NMR spectrum.

¹H-NMR (solvent: CDCl₃; reference substance: TMS), δ, ppm: 3.02 s (2CH₃), 4.82 m (2CH₂), 5.92 m (2H; —HC═CH—). This spectrum is identical to the spectrum of 2-butenyl 1,4-dimesylate described in the literature (H.-J. Lim et al., J. Org. Chem., Vol. 60, 1995, pp. 2326-2327).

### 19b) 1,4-Bis(trifluoromethoxy)but-2-ene CF₃OCH₂CH═CHCH₂OCF₃

CH₃SO₂OCH₂CH═CHCH₂OSO₂CH₃+2 KOCF₃→→CF₃OCH₂CH═CHCH₂OCF₃+2 KOSO₂CH₃

6.83 g (31.3 mmol) of trifluoromethyl triflate, CF₃SO₂OCF₃, are slowly added with stirring and cooling of the reaction mixture using a bath (0° C.) to 1.68 g (28.9 mmol) of dry potassium fluoride suspended in 31 ml of dry N,N-dimethylacetamide in a 100 ml round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 31 ml of dry N,N-dimethylacetamide and 2.9 g (11.9 mmol) of 2-butenyl 1,4-dimesylate from Example 15a) are added to the suspension of KOCF₃ remaining in the flask at a bath temperature of 0° C., and the reaction mixture is stirred at 75-80° C. (bath temperature) for 2 hours. The reaction mixture is subsequently cooled to 0° C. and diluted with cold (0° C.) water. The organic phase is separated off, and the aqueous phase is extracted four times with 10 ml of n-pentane. After drying using MgSO₄, the drying agent is filtered off, and n-pentane is removed in a rotary evaporator. The residue obtained in this way is re-condensed in vacuo. 2.40 g of a clear colourless liquid are obtained. Besides DMA and pentane, this comprises 34% of 1,4-bis(trifluoromethoxy)but-2-ene. The yield of 1,4-bis(trifluoromethoxy)but-2-ene is 30%, based on 2-butenyl 1,4-dimesylate. The product can be distilled in vacuo at 51 mbar (boiling point: 36° C./51 mbar). The product, 1,4-bis(trifluoromethoxy)but-2-ene, is characterised by means of ¹H- and ¹⁹F-NMR spectra. ¹H-NMR (solvent: CDCl₃; reference substance: TMS), δ, ppm: 4.54 m (2 CH₂), 5.83 m (2H, —CH═CH—). ¹⁹F-NMR (solvent: CDCl₃; reference substance: CCl₃F), δ, ppm: −60.7 s (2 OCF₃).

### Example 20

### 1-Bromo-4-trifluoromethoxybutane, BrCH₂CH₂CH₂CH₂OCF₃

### 20a) 4-Bromobutyl trifluoromethanesulfonate,

BrCH₂CH₂CH₂CH₂OSO₂CF₃

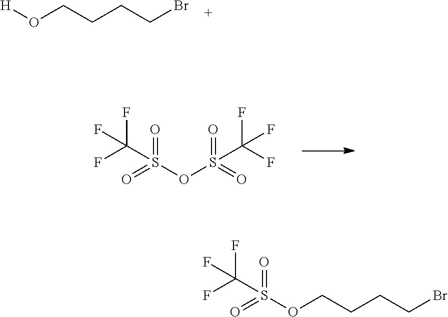

10.3 g of pyridine and 29.6 g of trifluoromethanesulfonic anhydride are added at 0° C. under a nitrogen atmosphere and with stirring to a solution of 15.3 g of 4-bromobutanol in 20 ml of dichloromethane, and the mixture is stirred at 0° C. for 0.5 hour and then at 20-25° C. for 2 hours. In order to isolate the product, the reaction mixture is cooled to 0° C., filtered through a layer of silica gel, and the solvent is removed from the filtrate at 15-25° C. in vacuo. A pale-yellow liquid forms, which is stored at −10-0° C. and should be reacted further within a few days.

MS: 285(M⁺)

### 20b) 1-Bromo-4-trifluoromethoxybutane

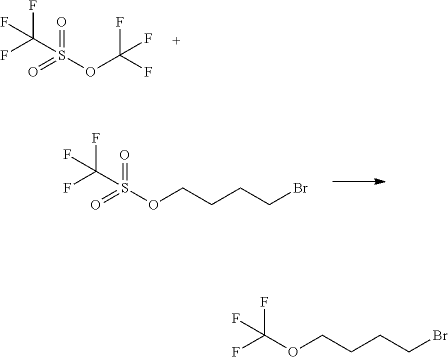

10.6 g of rubidium fluoride which has been pre-dried at 130° C. are added at −15° C. under nitrogen and with stirring to a solution of 21.8 g of trifluoromethyl trifluoromethanesulfonate in 50 ml of dry N,N-dimethylformamide (DMF) in a three-necked flask with dry-ice condenser, and the mixture is stirred at this temperature for 0.5 hour. Gaseous trifluoromethanesulfonyl fluoride and a DMF suspension of rubidium trifluoromethanolate form. 29.5 g of 4-bromobutyl trifluoromethanesulfonate are metered into the suspension, and the mixture is stirred at 20-30° C. for 4 hours. In order to isolate the product, the reaction mixture is added to ice, the organic phase is separated off at 0° C., and the aqueous phase is extracted with methyl tert-butyl ether (MTB ether). The combined organic phases are washed with water, the solvent is removed at 30° C. in vacuo, and the ether formed is chromatographed. A colourless, oily liquid forms.

MS: 221(M⁺)

### Example 21

### 1,3-Dibromo-2,2-bistrifluoromethoxymethylpropane

### 21a) 3-Bromo-2-bromomethyl-2-methanesulfonyloxymethylpropyl methanesulfonate

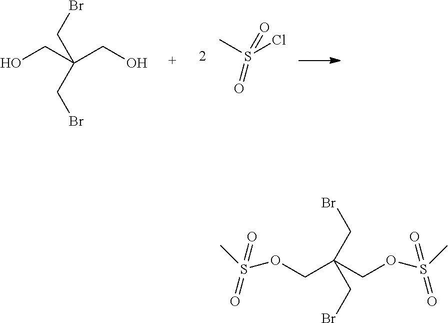

10.3 g of pyridine and 12.6 g of methanesulfonyl chloride are added at 0° C. under nitrogen and with stirring to a solution of 26.2 g of 2,2-bisbromomethylpropane-1,3-diol in 70 ml of dichloromethane, and the mixture is stirred at 0° C. for 0.5 hour and then at 20-25° C. for 6 hours. In order to isolate the product, the reaction mixture is cooled to 0° C. and filtered, and the filtrate is added to ice. The organic phase is separated off, dried using Na₂SO₄, and the solvent is removed at 10-20° C. in vacuo. A pale-yellow liquid forms, which is stored at −10-0° C. and should be reacted further within a few days.

### 21b) 1,3-Dibromo-2,2-bistrifluoromethoxymethylpropane

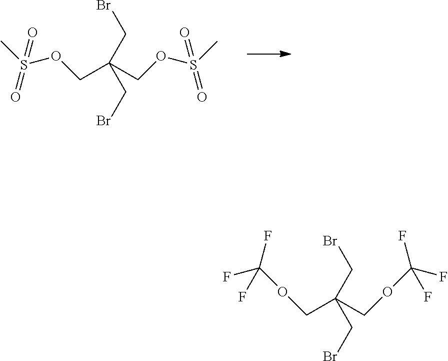

6.3 g of KF are added at −15° C. under nitrogen and with stirring to a solution of 21.8 g of trifluoromethyl trifluoromethanesulfonate in 50 ml of dry N,N-dimethylacetamide (DMA) in a three-necked flask with dry-ice condenser, and the mixture is stirred at this temperature for 0.5 hour. Gaseous trifluoromethanesulfonyl fluoride and a DMA suspension of potassium trifluoromethanolate form. 43.8 g of the dimesylate are metered into the suspension, and the mixture is stirred at 50-70° C. for 9 hours. In order to isolate the product, the reaction mixture is cooled to room temperature, added to ice, and the organic phase is separated off at 0° C. The aqueous phase is extracted with methyl tert-butyl ether (MTB ether), and the combined organic phases are washed with water. The solvent is removed from this phase at 30° C. in vacuo, and the product formed is chromatographed. A colourless, oily liquid forms.

MS: 398(M⁺)

### Example 22

### 3-Trifluoromethoxy-2-trifluoromethoxymethylpropene

### 22a) 2-Methanesulfonyloxymethylallyl methanesulfonate

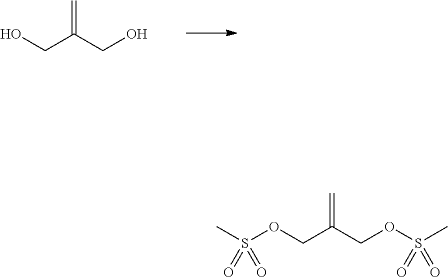

Analogously to the preparation of 3-bromo-2-bromomethyl-2-methanesulfonyloxymethylpropyl methanesulfonate, the diester 2-methanesulfonyloxymethylallyl methanesulfonate is obtained from 2-methylenepropane-1,3-diol and stored at −10-0° C. The diester should be reacted further within a few days.

### 22b) 3-Trifluoromethoxy-2-trifluoromethoxymethylpropene

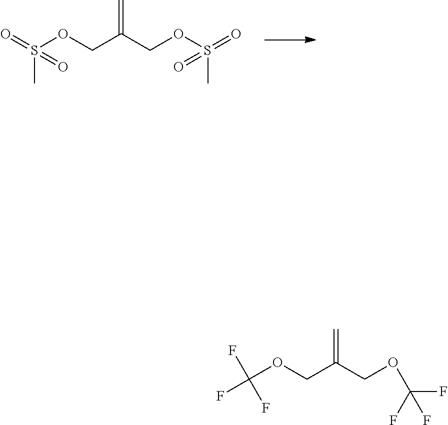

6.3 g of KF are at −15° C. under nitrogen and with stirring added to a solution of 21.8 g of trifluoromethyl trifluoromethanesulfonate in 50 ml of dry N,N-dimethylacetamide (DMA) in a three-necked flask with dry-ice condenser, and the mixture is stirred at this temperature for 0.5 hour. Gaseous trifluoromethanesulfonyl fluoride and a DMA suspension of potassium trifluoromethanolate form. 24.4 g of the dimesylate are metered into the suspension, and the mixture is stirred at 50-60° C. for 4 hours. In order to isolate the product, the reaction mixture is cooled to room temperature and added to ice. The organic phase is separated off at 0° C., and the remaining phase is distilled at atmospheric pressure. A colourless, mobile liquid forms.

MS: 224(M⁺)

### 22c) 3-Trifluoromethoxy-2-trifluoromethoxymethylpropene (2nd Route)

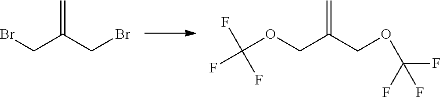

6.3 g of KF are added at −15° C. under nitrogen and with stirring to a solution of 21.8 g of trifluoromethyl trifluoromethanesulfonate in 50 ml of dry N,N-dimethylacetamide (DMA) in a three-necked flask with dry-ice condenser, and the mixture is stirred at this temperature for 0.5 hour. Gaseous trifluoromethanesulfonyl fluoride and a DMA suspension of potassium trifluoromethanolate form. 21.4 g of the dibromide are metered into the suspension, and the mixture is stirred at 50-60° C. for 24 hours. In order to isolate the product, the reaction mixture is cooled to room temperature and added to ice. The organic phase is separated off at 0° C., and the remaining phase is distilled at atmospheric pressure. A colourless, mobile liquid forms.

### Example 23

### 3-Trifluoromethoxypropan-1-ol

### 23a) 3-Vinyloxypropyl methanesulfonate

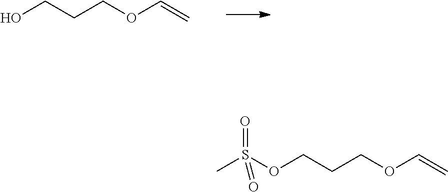

Analogously to the preparation of 3-bromo-2-bromomethyl-2-methanesulfonyloxymethylpropyl methanesulfonate, 3-vinyloxypropyl methanesulfonate is obtained and stored at −10-0° C. The ester should be reacted further within a few days.

### 23b) 3-Trifluoromethoxypropan-1-ol

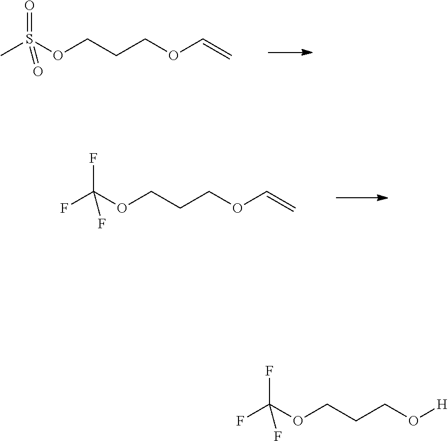

6.3 g of KF are added at −15° C. under nitrogen and with stirring to a solution of 21.8 g of trifluoromethyl trifluoromethanesulfonate in 50 ml of dry N,N-dimethylacetamide (DMA) in a three-necked flask with dry-ice condenser, and the mixture is stirred at this temperature for 0.5 hour. Gaseous trifluoromethanesulfonyl fluoride and a DMA suspension of potassium trifluoromethanolate form. 18.0 g of the mesylate are metered into the suspension, and the mixture is stirred at 50-70° C. for 12 hours. In order to isolate the product with parallel deprotection of the alcohol function, the reaction mixture is added to 0.5 N HCl which has been cooled to 0° C., and the mixture is stirred for 1 hour. The organic phase is then separated off, and the aqueous phase is extracted with methyl tert-butyl ether (MTB ether). The combined organic phases are washed with water and dried using Na₂SO₄. The solvent is removed at 30° C. in vacuo, and the alcohol formed is chromatographed. A colourless, oily liquid forms.

MS: 144(M⁺)

### Example 24

### Ethyl 2-trifluoromethoxylactate, CF₃OCH(CH₃)C(O)OC₂H₅

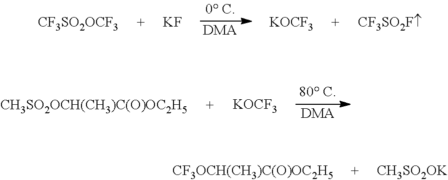

2.59 g (44.5 mmol) of dry potassium fluoride are suspended in 20 ml of dry N,N-dimethylacetamide (DMA) in a 100 ml round-bottomed flask with reflux condenser which is cooled to −80° C., and cooled to 0° C. 10.2 g (46.8 mmol) of trifluoromethyl triflate, CF₃SO₂OCF₃, are slowly added with stirring and cooling of the reaction mixture by means of a bath cooled to 0° C. The reaction mixture is kept at 0° C. for 1 hour. The temperature of the reflux condenser is then increased to room temperature in order to remove trifluoromethylsulfonyl fluoride, CF₃SO₂F, which is formed during the reaction. 6.9 g (35.2 mmol) of ethyl 2-mesyllactate, CH₃SO₂OC(CH₃)HC(O)OC₂H₅, are added to the suspension of KOCF₃ which remains, and the reaction mixture is stirred at 80° C. for 47 hours. The reaction mixture is then cooled to 0° C., and 30 ml of water are added. 10 ml of n-pentane are subsequently added, and the organic phase is separated off. The aqueous phase is extracted twice with n-pentane. The combined organic phases are dried using MgSO₄, and the pentane is distilled off after filtration, leaving 4.14 g of a liquid material which comprises 2.59 g of ethyl 2-trifluoromethoxylactate (62.6%), ethyl 2-mesyllactate (6.5%), DMA (8.9%) and n-pentane (22.0%). The yield of ethyl 2-trifluoromethoxylactate is 2.59 g (39%), based on ethyl 2-mesyllactate. The product, ethyl 2-trifluoromethoxylactate, is characterised by means of ¹H- and ¹⁹F-NMR spectra.

NMR data: ¹H-NMR (solvent: CD₃CN; reference substance: TMS), δ, ppm: 1.26 t (CH₃); ³J_{H,H}=7.2 Hz, 1.52 d (CH₃); ³J_{H,H}=6.9 Hz, 4.21 q (CH₂); ³J_{H,H}=7.2 Hz, 4.82 q (CH); ³J_{H,H}=6.9 Hz. ¹⁹F-NMR (solvent: CD₃CN; reference substance: CCl₃F), δ, ppm: −60.1 s (OCF₃).

### Example 25

### 2-Trifluoromethoxyacetic acid sodium salt (sodium 2-trifluoromethoxyacetate), CF₃OCH₂C(O)ONa

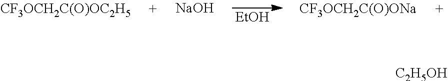

5.6 g (139.2 mmol) of sodium hydroxide are dissolved in 80 ml of dry ethanol in a 250 ml round-bottomed flask with reflux condenser. 25.3 g (146.8 mmol) of ethyl 2-trifluoromethoxyacetate are added thereto with stirring, and the solidifying mixture is heated at 70° C. for 18 hours. The solvent is then condensed off at 70° C. in vacuo, leaving 22.6 g of white solid, sodium 2-trifluoromethoxyacetate. The yield of sodium 2-trifluoromethoxyacetate is 22.6 g (98%), based on ethyl 2-trifluoromethoxyacetate. The substance decomposes above 200° C. without melting. The product, sodium 2-trifluoromethoxyacetate, is characterised by means of ¹H- and ¹⁹F-NMR spectra.

NMR data: ¹H-NMR (solvent: DMSO-D₆; reference substance: TMS), δ, ppm: 4.11 s (CH₂). ¹⁹F-NMR (solvent: DMSO-D₆; reference substance: CCl₃F), δ, ppm: −58.2 s (OCF₃).

### Example 26

### 2-Trifluoromethoxyacetic acid, CF₃OCH₂C(O)OH

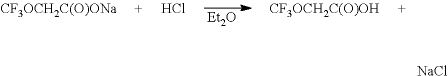

20.4 g (122.9 mmol) of sodium 2-trifluoromethoxyacetate from Example 21 are suspended in 100 ml of dry diethyl ether in a 250 ml round-bottomed flask with reflux condenser. Dry HCl gas is passed through the mixture in a 3- to 5-fold excess. A finely divided solid forms in the process. The reaction mixture is stirred at room temperature for 12 hours. All volatile products are then distilled off in vacuo at 10⁻³ mbar and firstly room temperature, later 70° C., into two distillation traps connected in series (−20° C. and −196° C.). The product collects in the front cold trap and gives, after fractional distillation, 15.0 g of clear colourless liquid, 2-trifluoromethoxyacetic acid. The yield of 2-trifluoromethoxyacetic acid is 85%, based on sodium 2-trifluoromethoxyacetate. The boiling point is 50.5° C. at 6 mbar. The melting point of the compound is 18-23° C., the density is about 1.5 g/cm³, and the pKₐ value is determined as 2.7 (25° C.). The product, 2-trifluoromethoxyacetic acid, is characterised by means of ¹H- and ¹⁹F-NMR spectra.

NMR data: ¹H-NMR (solvent: CD₃CN; reference substance: TMS), δ, ppm: 4.59 s (CH₂), 8.80 br. s (OH). ¹⁹F-NMR (solvent: CD₃CN; reference substance: CCl₃F), δ, ppm: −61.8 s (OCF₃).

### Example 27

### 1-Trifluoromethoxyhexan-6-ol, CF₃OCH₂(CH₂)₄CH₂OH

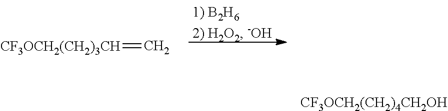

3.40 g (20.2 mmol) of 1-trifluoromethoxyhex-5-ene from Example 6d) are dissolved in 7 ml of dry diethyl ether in a 25 ml round-bottomed flask with reflux condenser and cooled to 0° C. by means of an ice bath. 0.19 g (6.9 mmol) of diborane is passed into this solution. When the introduction of gas is complete, the reaction mixture is stirred at room temperature for one hour and subsequently heated under reflux for 5 hours (bath temperature: 170° C.), during which the diethyl ether is distilled off. The reaction mixture is then cooled to room temperature and evacuated at about 50 mbar for 5 minutes. After aeration, 3 ml of water, 3 ml of 3 M sodium hydroxide solution and 3 ml of 30% hydrogen peroxide solution are added successively. After the mixture has been stirred for one hour, it is transferred into a separating funnel and washed three times with n-pentane. The combined organic phases are dried using MgSO₄, and, after filtration, the pentane is removed in a rotary evaporator, giving 3.48 g of a clear colourless liquid, whose composition is determined by means of ¹H-NMR as 93% of product and 7% of pentane. The yield of 1-trifluoromethoxyhexan-6-ol is 3.22 g (86%), based on 1-trifluoromethoxyhex-5-ene.

The product, 1-trifluoromethoxyhexan-6-ol, is characterised by means of ¹H- and ¹⁹F-NMR spectra.

NMR data: ¹H-NMR (solvent: CD₃CN; reference substance: TMS), δ, ppm: 1.21-1.44 m (4H, 2 (CH₂), 1.49 quin. (CH₂), 1.69 quin. (CH₂), 2.51 t (OH); ³J_{H,H}=5.3 Hz, 3.48 q (CH₂OH), 4.03 t (CH₂—OCF₃); ³J_{H,H}=6.5 Hz. ¹⁹F-NMR (solvent: CD₃CN; reference substance: CCl₃F), δ, ppm: −61.0 s (OCF₃).

Note: Diborane is generated as follows: 0.39 g (10.3 mmol) of sodium borohydride in 10 ml of diethylene glycol dimethyl ether is added dropwise to a solution of 2.95 g (20.8 mmol) of boron trifluoride etherate in 5 ml of diethyl ether in a 25 ml round-bottomed flask. After the dropwise addition and after the evolution of gas is complete, the reaction mixture is, for the generation of the diborane, heated at 60° C. for a further 1 hour in order to complete the reaction.

### Example 28

### 2-(Trifluoromethoxy)acetophenone, CF₃OCH₂C(O)C₆H₅

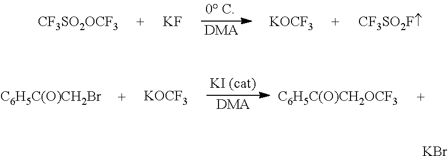

6.36 g (109.5 mmol) of dry potassium fluoride are suspended in 20 ml of dry N,N-dimethylacetamide (DMA) in a 500 ml round-bottomed flask with reflux condenser which is cooled to −80° C., and cooled to 0° C. 26.96 g (123.6 mmol) of trifluoromethyl triflate, CF₃SO₂OCF₃, are slowly added with stirring and cooling of the reaction mixture by means of a bath cooled to 0° C. The reaction mixture is kept at 0° C. for 1 hour. The temperature of the reflux condenser is then increased to room temperature in order to remove the trifluoromethylsulfonyl fluoride, CF₃SO₂F, which is formed during the reaction. 17.22 g (86.5 mmol) of 2-bromoacetophenone, BrCH₂C(O)C₆H₅, and 1.54 g (9.3 mmol) of potassium iodide are added to the suspension of KOCF₃ which remains, and the reaction mixture is stirred at room temperature for 92 hours. The reaction mixture is then filtered, and the filter cake is rinsed three times with 50 ml of n-pentane each time. 50 ml of water are added to the filtrate, and the organic phase which forms is separated off, washed twice with water and dried using MgSO₄. The solution is subsequently filtered, and the solvent is evaporated. The residue is subjected to fractional distillation (boiling point: 51-52° C. at 0.7 mbar). 8.1 g of liquid 2-(trifluoromethoxy)acetophenone are obtained (purity: 95%). The yield of 2-(trifluoromethoxy)acetophenone is 44%, based on 2-bromoacetophenone. The product, 2-(trifluoromethoxy)acetophenone, is characterised by means of ¹H- and ¹⁹F-NMR spectra.

NMR data: ¹H-NMR (solvent: CD₃CN; reference substance: TMS), δ, ppm: 5.40 s (CH₂), 7.55 t (2H, meta-H, C₆H₅), 7.68 t (1H, para-H, C₆H₅), 7.93 d (2H, ortho-H, C₆H₅). ¹⁹F-NMR (solvent: CD₃CN; reference substance: CCl₃F), δ, ppm: −61.1 s (OCF₃).

### Example 29

### 1-Trifluoromethoxyprop-2-yne, CF₃OCH₂C≡CH

C₆H₅SO₂OCH₂≡CH+KOCF₃→CF₃OCH₂C≡CH+C₅H₆SO₂OK

21.30 g (97.7 mmol) of trifluoromethyl triflate, CF₃SO₂OCF₃, are slowly added with stirring and cooling of the reaction mixture using a bath (0° C.) to 5.36 g (92.3 mmol) of dry potassium fluoride suspended in 90 ml of dry N,N-dimethylacetamide (DMA) in a 250 ml round-bottomed flask with reflux condenser which is cooled to −80° C. The reaction mixture is stirred at 0° C. for a further 1 hour. The temperature of the reflux condenser is then increased to room temperature in order to remove the trifluoromethylsulfonyl fluoride, CF₃SO₂F, formed during the reaction over the course of 30 min. 15.10 g (76.6 mmol) of prop-2-yn-1-yl benzenesulfonate are added to the suspension of KOCF₃ remaining in the flask at a bath temperature of 0° C., and the reaction mixture is stirred at 75° C. (bath temperature) for 16 hours. The product formed is then condensed in a trap cooled by means of liquid nitrogen by applying a vacuum. 7.52 g of a mixture of DMA and the desired product are obtained. According to ¹H-NMR, the mixture comprises 4.88 g (39.0 mmol) of 1-trifluoromethoxyprop-2-yne. 69 ml of water are subsequently added to the reaction mixture, and volatile products are condensed off further in vacuo at 75° C. in a trap cooled by means of liquid nitrogen. An additional 9.10 g of a mixture of DMA, water and the desired product are obtained. 5.94 g of 1-trifluoromethoxyprop-2-yne are obtained from the two mixtures on distillative separation. The boiling point of 1-trifluoromethoxyprop-2-yne is 34° C. The yield is 62%, based on prop-2-yn-1-yl benzenesulfonate.

The product, 1-trifluoromethoxyprop-2-yne, is characterised by means of ¹H- and ¹⁹F-NMR spectra.

¹H-NMR (solvent: CD₃CN; reference substance: TMS), δ, ppm: 2.99 t (CH), 4.74 d (2H, CH₂), ⁴J_{H,H}=2.4 Hz. ¹⁹F-NMR (solvent: CD₃CN; reference substance: CCl₃F), δ, ppm: −61.2 s (OCF₃).

1. Process for the preparation of compounds containing at least one CF₃O group, comprising at least the reaction of CF₃O⁻ salts with compounds of the formula (I)
XₘCHₙ(LₒY)ₚ (I)
where:
X=—Cl, —Br, —I, —OR, —SR, —C(O)R, —C(O)OR, —H, —CN, —CR¹═CR²₂, —C≡CR² or —(CR³R⁴)_{q}X,
Y=—Hal, —OSO₂(CF₂)_{z}F, —OSO₂C_{z}H_{2z+1}, —OSO₂F, —OSO₂Cl, —OC(O)CF₃, or —OSO₂Ar,
L=independently of one another a single bond or linear or branched (CR³R⁴)_{q}-alkyl, optionally containing at least one aromatic ring, cycloalkyl, heterocyclic ring, O atom, S atom, double bond, triple bond and/or group X in the chain and/or in the side chain,
Ar=substituted or unsubstituted aryl,
m=1-2
n=0-2
o=0 or 1
p=1-3
q=1 to 20,
m+n+p=4
z=1-10,
R=aryl or cycloalkyl or alkylaryl (for example benzyl), optionally substituted by at least one fluorine and/or chlorine and/or bromine and/or iodine atom and/or other functional group (such as, for example, NO₂, NH₂, CN, C(O)R, C(O)OR, C(O)NR₂), linear or branched H(CR³R⁴)ᵣ-alkyl (r=1 to 20), optionally containing at least one aromatic ring, heterocyclic ring, O atom, S atom, double bond and/or triple bond and optionally substituted by at least one fluorine and/or chlorine atom,
R¹, R², R³ and R⁴=independently of one another H, aryl, cycloalkyl, optionally substituted by at least one fluorine and/or chlorine atom, linear or branched alkyl, optionally containing at least one aromatic ring, heterocyclic ring, O atom, S atom, double bond and/or triple bond and optionally substituted by at least one fluorine and/or chlorine atom,
and CHₙ and Lₒ may together form a cycloalkyl or aromatic ring or heterocyclic ring.

2. Process according to claim 1, characterised in that use is made of compounds of the formula (I) where
n=2, o=0 or 1, p=1, q=1
Y=—Hal, —OSO₂CF₃, or —OSO₂CH₃,
X=—Cl, —Br, —I, or —CR¹═CR²₂,
L=linear or branched (CR³R⁴)_{q}-alkyl, optionally containing at least one O atom,
R¹ and R²=independently of one another H or methyl, and
R³ and R⁴=independently of one another linear or branched C1-C6 alkyl.

3. Process according to claim 1, characterised in that (R⁵)₄N⁺CF₃O⁻ salts are used, in which R⁵ can be, independently of one another, C1-C4-alkyl.

4. Process for the preparation of compounds containing at least one CF₃O group, comprising the use of KOCF₃ and/or RbOCF₃, where KOCF₃ and RbOCF₃ are formed in situ or RbOCF₃ is added separately.

5. Process according to claim 4, characterised in that KOCF₃ and/or RbOCF₃ is/are reacted with compounds containing at least one group Y, in which Y=—Hal, —OSO₂CH₃, or —OSO₂Ar.

6. Process according to claim 4, characterised in that compounds of the formula (I) are used.

7. Process according to claim 6, characterised in that use is made of compounds of the formula (I) where
n=2, o=0 or 1, p=1, q=1
Y=Hal, —OSO₂CF₃, or —OSO₂CH₃,
X=Cl, Br, I, or —CR¹═CR²₂,
L=linear or branched (CR³R⁴)_{q}-alkyl, optionally containing at least one O atom,
R¹ and R²=independently of one another H or methyl, and
R³ and R⁴=independently of one another linear or branched C1-C6 alkyl.

8. Process according to claim 4, characterised in that KOCF₃ or RbOCF₃ is prepared by reaction of KF or RbF with trifluoromethyl triflate.

9. Compound of the formula (II)
XₘCHₙ(LₒOCF₃)ₚ (II)
Where:
X=—Cl, —Br, —I, —OR, —SR, —C(O)R, —C(O)OR, —H, —CN, —CR¹═CR²₂, —C≡CR² or —(CR³R⁴)_{q}X,
L=independently of one another a single bond or linear or branched (CR³R⁴)_{q}-alkyl, optionally containing at least one aromatic ring, cycloalkyl, heterocyclic ring, O atom, S atom, double bond, triple bond, and/or group X in the chain and/or in the side chain,
m=1-2
n=0-2
o=1
p=1-3
q=2 to 20,
m+n+p=4
R=aryl or cycloalkyl or alkylaryl (for example benzyl), optionally substituted by at least one fluorine and/or chlorine and/or bromine and/or iodine atom and/or other functional group (such as, for example, NO₂, NH₂, CN, C(O)R, C(O)OR, C(O)NR₂), linear or branched H(CR³R⁴)ᵣ-alkyl (r=1 to 20), optionally containing at least one aromatic ring, heterocyclic ring, O atom, S atom, double bond and/or triple bond and optionally substituted by at least one fluorine and/or chlorine atom,
R¹, R², R³ and R⁴=independently of one another H, aryl, cycloalkyl, optionally substituted by at least one fluorine and/or chlorine atom, linear or branched alkyl, optionally containing at least one aromatic ring, heterocyclic ring, O atom, S atom, double bond and/or triple bond and optionally substituted by at least one fluorine and/or chlorine atom,
and CHₙ and Lₒ may together form a cycloalkyl or aromatic ring or heterocyclic ring,
where the compounds CF₃O—(CH₂—CH₂—O)₂—OCF₃ and C₂H₅O—(CH₂—CH₂—O)₂—C₂H₅—OCF₃ are excluded.

10. Compounds according to claim 9, characterised in that the variables X and L have the following meanings:
X=—Cl, —Br, —I, —OR, —CR¹═CR²₂ or —(CR³R⁴)_{q}X
L=independently of one another linear or branched (CR³R⁴)_{q}-alkyl containing at least one aromatic ring, cycloalkyl, heterocyclic ring, S atom, triple bond, and/or group X, apart from X=halogen, in the chain and/or in the side chain.

11. A process for the preparation of surface-active compounds containing CF₃O groups comprising using a compound according to claim 9.
